SEQUENCE LISTING (20 sequences)

SEQ ID NO: 1 - protein, 195 aa, Bos sp.
  REGION 1..195: note = Description of sequence: IFNT Clone BTP509
MAFVLSLLMALVLVSYGQGRSLGCYLSEDHMLGARENLRLLARMNRLSPHPCLQDRKDFGLPQEMVEGNQLQKDQAISVLHEMLQQCLNLFYTEHSSAAWNTTLLEQLCTGLQQQLEDLDACLGPVMGEKDSDMGRMGPILTVKKYFQGIHVYLKEKEYSDCAWEIIRVEMMRALSSSTTLQKRLRKMGGDLNSL

SEQ ID NO: 2 - DNA, 1033 bp, Bos sp.
  misc_feature 1..1033: note = Description of sequence: IFNT Clone BTP509
  source 1..1033: mol_type = genomic DNA
gatccccggaaactagaattcacctgaaggttcacccagaccccatctcagccagcccagcagcagccacatcttccccatggccttcgtgctctctctactgatggccctggtgctggtcagctacggccagggacgatctctgggttgttacctgtctgaggaccacatgctaggtgccagggagaacctcaggctcctggcccgaatgaacagactctctcctcatccctgtctgcaggacagaaaagactttggtcttcctcaggagatggtggagggcaaccagctccagaaggatcaggctatctctgtgctccacgagatgctccagcagtgcctcaacctcttctacacagagcactcgtctgctgcctggaacaccaccctcctggagcagctctgcactgggctccaacagcagctggaggacctggacgcctgcctgggcccagtgatgggagagaaagactctgacatgggaaggatgggccccattctgactgtgaagaagtacttccagggtatccatgtctacctgaaagaaaaagaatacagtgactgcgcctgggaaatcatcagagtggagatgatgagagccctctcttcatcaaccaccttgcaaaaaaggttaagaaagatgggtggagatctgaactcactttgagatgactctcgctgactaagatgccacatcaccttcgtacactcacctgtgttcatttcagaagactctgatttctgcttcagccaccgaaatcattgaattactttaactgatactttgtcagcagtaataagcaagtagatataaaagtactcagctgtaggggcatgagtccttaagtgatgcctgccctgatgttatctgttgttgatttatgtattccttcttgcatctaacatacttaaaatattaggaaatttgtaaagttacatttcatttgtacatctattaaaatttctaaaacatgtttaccattttgtgttattaaatttgtcctttgttctatttattaaatcaaagaaaatc

SEQ ID NO: 3 - protein, 195 aa, Bos sp.
  REGION 1..195: note = Description of sequence: IFNT
MAFVLSLLMALVLVSYSPGRSLGCYLSENHMLGARENLRLLAQMNRLSTHSCLQDRKDFGLPWEMVEGDQLQKDQAISVLHEMLQQCFNLFHTEHSSAAWNTTLLEQLCTGLHQQLDDLDACLGQVMEEKDSALGRMGPILTVKKYFQGIHVYLKKKEYSDCAWEIIRVEMMRALSSSTSLQERLRKIGGDLNSS

SEQ ID NO: 4 - DNA, 588 bp, Bos sp.
  misc_feature 1..588: note = Description of sequence: IFNT
  source 1..588: mol_type = genomic DNA
atggcttttgtgctctctctactgatggccttggtgctggtcagctacagcccgggacgatctctgggttgttacctgtctgagaaccacatgctaggtgccagggagaacctcaggctcctggcccaaatgaacagactctccactcattcctgtctgcaagacagaaaagactttggtcttccctgggagatggtggagggtgaccagctccagaaggaccaggctatctctgtgctccacgagatgctccagcagtgcttcaacctcttccacacagagcactcgtctgctgcctggaacaccaccctcctggagcagctctgcactggactccatcagcagctggatgacctggatgcctgcctggggcaggtgatggaagagaaagactctgccctgggaaggatgggccccattctgaccgtgaagaagtacttccagggcatccatgtgtacctgaaaaagaaggaatacagcgactgcgcctgggaaatcatcagagtggagatgatgagagccctctcttcatcaaccagcttgcaagaaaggttaagaaagataggtggagatctgaactcatcttga

SEQ ID NO: 5 - protein, 195 aa, Bos sp.
  REGION 1..195: note = Description of sequence: IFNT2
MAFVLSLLMALVLVSYGPGRSLGCYLSEDHMLGARENLRLLARMNRLSPHPCLQDRKDFGLPQEMVEGNQLQKDQAISVLHEMLQQCLNLFYTEHSSAAWNTTLLEQLCTGLQQQLEDLDACLGPVMGEKDSDMGRMGPILTVKKYFQGIHVYLKEKEYSDCAWEIIRVEMMRALSSSTTLQKRLRKMGGDLNSL

SEQ ID NO: 6 - DNA, 1313 bp, Bos sp.
  misc_feature 1..1313: note = Description of sequence: IFNT2
  source 1..1313: mol_type = genomic DNA
tttatttagtttctcatttaattgatatacatttacattgacaaacccaaattttattgggaaaattaaatttctactgtaaaaattaagagtttagattgactacatttcctaggtcaaacagaaaatatctaactgaaaacacaaacaggaagtgagagagaaattttcggataatgagtaccgtcttccctatttaaaagccttgcttagaacgatcatcatcagagaacctacctgaaggttcacccagaccccatctcagccagcccagcagcagccacatcttccccatggccttcgtgctctctctactgatggccctggtgctggtcagctacggcccgggacgatctctgggttgttacctgtctgaggaccacatgctaggtgccagggagaacctcaggctcctggcccgaatgaacagactctctcctcatccctgtctgcaggacagaaaagactttggtcttcctcaggagatggtggagggcaaccagctccagaaggatcaggctatctctgtgctccatgagatgctccagcagtgcctcaacctcttctacacagagcactcgtctgctgcctggaacaccaccctcctggagcagctctgcactgggctccaacagcagctggaggacctggacgcctgcctgggcccagtgatgggagagaaagactctgacatgggaaggatgggccccattctgactgtgaagaagtacttccagggtatccatgtctacctgaaagaaaaagaatacagtgactgcgcctgggaaatcatcagagtggagatgatgagagccctctcttcatcaaccaccttgcaaaaaaggttaagaaagatgggtggagatctgaactcactttgagatgactctcgctgactaagatgccacatcaccttcgtacactcacctgtgttcatttcagaagactctgatttctgcttcagccaccgaaatcattgaattactttaactgatactttgtcagcagtaataagcaagtagatataaaagtactcagctgtaggggcatgagtccttaagtgatgcctgccctgatgttatctgttgttgatttatgtattccttcttgcatctaacatacttaaaatattaggaaatttgtaaagttacatttcatttgtacatctattaaaatttctaaaacatgtttaccattttgtgttattaaatttgtcctttgttctatttattaaatcaaagaaaatgagtttctttactcaaaaactttattattattattattattaaaactttattaaagaatgggtggtt

SEQ ID NO: 7 - protein, 195 aa, Bos sp.
  REGION 1..195: note = Description of sequence: IFNT3
MAFVLSLLMALVLVSYGPGRSLGCYLSEDHMLGARENLRLLARMNRLSPHPCLQDRKDFGLPQEMVEGSQLQKDQAISVLHEMLQQCFNLFHIEHSSAAWNTTLLEQLCTGLQQQLEDLDACLGPVMGEKDSDMGRMGPILTVKKYFQDIHVYLKEKEYSDCAWEIIRVEMMRALSSSTTLQKRLRKMGGDLNSL

SEQ ID NO: 8 - DNA, 1073 bp, Bos sp.
  misc_feature 1..1073: note = Description of sequence: IFNT3
  source 1..1073: mol_type = genomic DNA
ctgaaggttcacccagaccccatctcagccagcccagcagcagccacatcttccccatggccttcgtgctctctctactgatggccctggtgctggtcagctacggcccgggacgatctctgggttgttacctgtctgaggaccacatgctaggtgccagggagaacctcaggctcctggcccgaatgaacagactctctcctcatccctgtctgcaggacagaaaagactttggtcttcctcaggagatggtggagggcagccagctccagaaggatcaggctatctctgtgctccacgagatgctccagcagtgcttcaacctcttccacatagagcactcgtctgctgcctggaacaccaccctcctggagcagctctgcactgggctccaacagcagctggaggacctggacgcctgcctgggcccagtgatgggagagaaggactctgacatgggaaggatgggccccattctgactgtgaagaagtacttccaggacatccatgtctacctgaaagaaaaggaatacagtgactgcgcctgggaaatcatcagagtggagatgatgagagccctctcttcatcaaccaccttgcaaaaaaggttaagaaagatgggtggagatctgaactcactttgagatgactctcgctgactaagatgccacatcaccttcgtacactcacctgtgttcatttcagaagactctgatttctgcttcagccaccgaattcattgaattactttagccgatactttgtcagcagtaataagcaagtagatataaaagtactcagctgtaggggcatgagtccttaagtgatgcctgccctgatgttatctgttgttgatttatgtattccttcttgcatctaacatacttaaaatattaggatatttgtaaagttacatttcatttgtacatctattaaaatttctaaaacatgtttaccattttgtgttattaaatttgtcctttgttctatttattaaatcaaagaaaatgagtttctttactcaaaaactttattattattattaaaactttattaaagaaaaaaaaaaaaaa

SEQ ID NO: 9 - protein, 4 aa, Bos sp.
ENLR

SEQ ID NO: 10 - protein, 4 aa, Bos sp.
LLDR

SEQ ID NO: 11 - protein, 13 aa, Bos sp.
MNRPSPHSCLQDR

SEQ ID NO: 12 - protein, 8 aa, Bos sp.
MDPIVTVK

SEQ ID NO: 13 - protein, 12 aa, Bos sp.
YFQGIHDYLQEK

SEQ ID NO: 14 - protein, 5 aa, Bos sp.
VEMMR

SEQ ID NO: 15 - protein, 9 aa, Bos sp.
ALTSSTTLK

SEQ ID NO: 16 - protein, 4 aa, Bos sp.
LLAR

SEQ ID NO: 17 - protein, 10 aa, Bos sp.
LSPHPCLQDR

SEQ ID NO: 18 - protein, 8 aa, Bos sp.
MGPILTVK

SEQ ID NO: 19 - protein, 10 aa, Bos sp.
YFQGIHVYLK

SEQ ID NO: 20 - protein, 10 aa, Bos sp.
ALSSSTTLQK

(19) United States
(12) Patent Application Publication
(10) Pub. No.: US 2026/0191633 A1
(43) Pub. Date: Jul. 9, 2026

(54) OPEN COW TEST

(71) Applicant: COLORADO STATE UNIVERSITY RESEARCH FOUNDATION, Fort Collins, CO (US)

(72) Inventors: Thomas R. Hansen, Fort Collins, CO (US); Jeanette V. Bishop, Fort Collins, CO (US); Hana Van Campen, Fort Collins, CO (US); Aydin Guzeloglu, Fort Collins, CO (US)

(21) Appl. No.: 19/129,844

(22) PCT Filed: Nov. 15, 2023
(86) PCT No.: PCT/US2023/079746

§ 371 Date: May 14, 2025

Related U.S. Application Data
(60) Provisional application No. 63/383,746, filed on Nov. 15, 2022.

(51) Int. Cl. A61D 17/00; C12Q 1/6888; G01N 33/68; G01N 33/74

(52) U.S. Cl. CPC A61D 17/006; C12Q 1/6888; G01N 33/6866; G01N 33/689; G01N 33/743; G01N 2333/555; G01N 2333/723; G01N 2440/38

(57) ABSTRACT

In some aspects, disclosed herein is a method of determining pregnancy and identifying open cows, comprising breeding a cow, obtaining a genital tract sample from the cow, detecting a level of an interferon tau (IFNT) polypeptide in the genital tract sample; identifying the cow as pregnant if the level of IFNT is higher than a reference control or as not pregnant if the level of IFNT is the same as or lower than the reference control, and performing additional breeding of the cow if the cow is determined to be not pregnant.

### CROSS-REFERENCE TO RELATED APPLICATIONS

This PCT application claims priority to, and the benefit of, U.S. Provisional Patent Application No. 63/383,746, filed Nov. 15, 2022, which is incorporated by reference herein in its entirety.

### STATEMENT REGARDING FEDERALLY SPONSORED RESEARCH

This invention was made with government support under grant 2016-31200-06008 awarded by the United States Department of Agriculture. The government has certain rights in the invention.

### REFERENCE TO SEQUENCE LISTING

The sequence listing submitted on Nov. 15, 2023, as an .XML file entitled “10975-040WO1_ST26.xml” created on Nov. 8, 2023, and having a file size of 25,905 bytes is hereby incorporated by reference pursuant to 37 C.F.R. § 1.52(e)(5).

### FIELD

The present disclosure relates to early determination of pregnancy in cows.

### BACKGROUND

Dairy cattle are plagued with infertility and a high incidence of embryo mortality in part due to the metabolic demands of milk production. Early embryo mortality and failure to conceive at first service contributes to subfertility costing the dairy cattle industry an annual $1.6 billion loss in the United States of America and $1.28 trillion worldwide. Dairy cows in the US have pregnancy rates of only 30 to 50% on their first breeding by artificial insemination (AI) following calving. Most pregnancy losses are caused by embryo mortality that occurs before day 21 of pregnancy. Because of 30-40% pregnancy rates at each AI service, it can take up to 5 AI cycles for cows to become pregnant. This has the most detrimental impact on milk production per year in year 2 after these cows calve. Given the limitations described above, there is a need to develop methods for early identification of non-pregnant or “open” cows following artificial insemination so that they can be managed earlier during lactation.

The methods disclosed herein address these and other needs.

### SUMMARY

The present disclosure relates to methods for early determination of pregnancy in a cow (i.e., an “open cow test” or “OCT”).

In some aspects, disclosed herein is a method of determining pregnancy and identifying open cows, comprising breeding a cow, obtaining a genital tract sample from the cow, detecting a level of an interferon tau (IFNT) polypeptide in the genital tract sample; identifying the cow as pregnant if the level of IFNT is higher than a reference control or as not pregnant if the level of IFNT is the same as or lower than the reference control, and performing additional breeding of the cow if the cow is determined to be not pregnant.

In some aspects, disclosed herein is a method of discriminating between a pregnant cow and an open cow, the method comprising breeding a cow, obtaining a genital tract sample from the cow, measuring a level of an interferon tau (IFNT) polypeptide in the genital tract sample, detecting a high IFNT level in the pregnant cow relative to a reference control, and detecting a low IFNT level in the open cow relative to a reference control, wherein the open cow is subjected to an additional round of breeding.

In some embodiments, the genital tract sample is obtained from the cow on or before day 18 post breeding. In some embodiments, the genital tract specimen is obtained from the open cow on day 15, 16, 17, or 18 post breeding.

In some embodiments, the method identifies the open cow on or before day 18 post breeding. In some embodiments, the methods identifies the open cow on day 15, 16, 17, or 18 post breeding. In some embodiments, the method detects the level of the IFNT polypeptide on or before day 18 post breeding. In some embodiments, the method detects the level of the IFNT polypeptide on day 15, 16, 17, or 18 post breeding.

In some embodiments, the level of the IFNT polypeptide is detected by an immunoassay. In some embodiments, the immunoassay comprises enzyme-linked immunoassay (ELISA), lateral flow immunosorbent assay, radioimmunoassay, or a derivative thereof. In some embodiments, the immunoassay is performed by contacting the genital tract specimen with an antibody targeting a glycosylated IFNT polypeptide.

In some embodiments, the glycosylated IFNT polypeptide comprises a bovine IFNT polypeptide. In some embodiments, the glycosylated IFNT polypeptide comprises a glycosyl molecule bound to an amino acid of the IFNT polypeptide. In some embodiments, the amino acid is located at position 101 of the IFNT polypeptide.

In some embodiments, the method detects an IFNT polypeptide comprising SEQ ID NO: 1, SEQ ID NO: 3, SEQ ID NO: 5, SEQ ID NO: 7, or a variant thereof.

In some embodiments, the method further comprises detecting a level of an additional RNA or polypeptide. In some embodiments, the additional RNA or polypeptide is interferon-stimulated gene 15 (ISG15) RNA or polypeptide in the genital tract sample.

In some embodiments, the method further comprises detecting a level of progesterone in a genital tract sample, blood sample, or a milk sample.

### BRIEF DESCRIPTION OF FIGURES

The accompanying figures, which are incorporated in and constitute a part of this specification, illustrate aspects described below.

FIG. 1 shows IFNT ELISA antigen specificity. The bovine IFNT ELISA detected IFNT and not other related IFNs at concentrations as high as 1,000 pg/ml. Note that IFNT absorbance values for IFNs other than IFNT were at 0 when adjusted for background (LOD) in the assay.

FIG. 2 shows detection of IFNT in blind vaginal swabs in cows on day 19 that were subsequently found to be open or pregnant on day 32 following AI using ultrasound. Accuracy estimates were based on calling cattle open with IFNT concentrations≤the Limit of Detection (LOD) and calling cows pregnant with IFNT concentrations≥the LOD for this assay.

FIG. 3 shows IFNT concentrations in blind vaginal swab samples collected from first calf heifers and lactating cows on days 0 and 13-19 based on retrospective ultrasound analysis for pregnancy on day 32 following AI. Accuracy estimates were based on calling cattle open with IFNT concentrations≤the LOD and calling cattle pregnant with IFNT concentrations≥the LOD for this assay.

FIG. 4 shows IFNT concentrations in blind vaginal swab samples collected from lactating cows on days 0 and 13-19 based on retrospective ultrasound analysis for pregnancy status on day 32 following AI. Notice that these data are a subset of all heifers and cows analyzed in FIG. 3 and illustrate IFNT profiles in open and pregnant lactating dairy cows. Removal of data from the first calf heifers significantly improved the accuracy and earlier detection of IFNT by day 16 following AI in Experiment 2. Accuracy values were based on calling cows open with IFNT concentrations<the LOD and pregnant cows with IFNT concentrations>the LOD for this assay.

FIG. 5 illustrates using transrectal palpation to guide the swab sampling device to the external os of the cervix (top illustration), a site of possibly greater IFNT concentrations and day 18 IFNT results (bottom graph). After the swab device is positioned adjacent to the external os of the cervix, the device is opened and then a cotton swab is pushed through the end of the collection device. Once it is in place, the device is “twirled around” for about 20-30 seconds for the cotton swab to be saturated with cervical/vaginal fluid and then it is retracted into the plastic sheath before removing from the vagina. Accuracy estimates were based on calling cows open with IFNT concentrations≤the LOD and pregnant cows with IFNT concentrations≥the LOD for this assay.

FIG. 6 shows IFNT concentrations in guided external cervical swab samples collected from cows on days 15-21 following AI based on retrospective ultrasound on day 32. Slightly elevated, but not significant mean levels of IFNT on days 18 and 20 represent cows that may have had some IFNT release on these days that contributed to the overall mean, but then lost their embryos prior to ultrasound (embryo mortality). This difference in IFNT on days 18 and 20 compared to day 32 ultrasound results is reflected in the False Positive cows. These data reflect all cattle (first calf heifers and older lactating cows) and are a significant improvement compared to similar cattle represented in FIG. 3. Accuracy estimates were based on calling cows open with IFNT concentrations≤the LOD and pregnant cows with IFNT concentrations≥the LOD for this assay.

FIG. 7 shows IFNT concentrations in guided external cervical swabs in lactating dairy cows on days 13 to 31 following AI. These data confirm detection of IFNT as early as day 15 and as late as day 25 using external os cervical swabs. IFNT data in FIG. 7 were compared to progesterone concentrations in serum on days 19, 21 and 31 following AI to determine if use of both markers improves accuracy when finding open cows (FIG. 8).

FIGS. 8A, 8B, 8C, 8D, and 8E show the IFNT concentrations in swab samples on days 13-31 and serum progesterone concentrations on days 19, 21 and 31 with ultrasound for pregnancy status on day 32. FIG. 8A describes cows that were correctly called open by Day 17 IFNT ELISA, Day 21 serum progesterone and day 32 ultrasound (10/34 cows). FIG. 8B describes cows that were accurately called open by Day 17 IFNT ELISA and day 32 ultrasound but were falsely called pregnant based on high serum progesterone on day 21 (7 cows out of 34 tested. The serum progesterone concentration was high on day 21. Open cows accurately identified by the ELISA and ultrasound totaled 17 out of the 34 cows tested. FIG. 8C represents open cows based on low IFNT on day 17, with high serum progesterone on day 21 and presence of a pregnancy on day 32 ultrasound. These false negative (FN) cows based on IFNT ELISA represented 5/34 cows. FIG. 8D represents cows called pregnant on Day 17 based on detection of IFNT, high Day 21 serum progesterone concentration and detection of pregnancy on Day 32 ultrasound (11 cows out of 34). FIG. 8E represents cows called pregnant on day 17, when in fact they were open based on low serum progesterone concentrations on day 21 and lack of a pregnancy detected by day 32 ultrasound. Notice that the threshold in detecting IFNT was low in this panel compared to FIG. 8D. This represents very low levels of IFNT reflecting a dying embryo (embryo mortality). This explains that loss of progesterone as well due to failure of maternal recognition of pregnancy. Many of these false positive cows are removed by raising the threshold of limits of detection. Use of both the OCT on days 17-19 coupled with rapid serum progesterone on day 21 is an approach to improving accuracy of diagnosing open cows early after AI.

FIG. 9 shows a comparison of IFNT with PSPB concentrations in cervical swab samples collected on days 13-31 following AI. Pregnancy status was determined by ultrasound on day 32 following AI. Note that IFNT was detected in swab samples by day 15, whereas there was not a significant increase in PBSP in open compared to pregnant cows in any of these samples tested up to day 31 of pregnancy.

FIG. 10 shows a cow-side lateral flow positive test (A) and a negative test (B) for bIFNT. The greatest utility for this diagnostic is to identify the cows that are negative for bIFNT and that are not pregnant (called open in the industry).

FIG. 11 shows rebreeding strategies for organic (A) and conventional dairy farms (B) when using OCT. Organic dairies use estrous detection and pedometer activity tracking to make decisions on when to AI. By using OCT on days 17, 18 or 19, cows are sorted and managed more intensively. Testing IFNT in swabs using a Day 18 OCT is shown. The OCT approach improves in context of open cow diagnostic when paired with another marker on days shown such as serum progesterone (S-P). AI1=first AI service. AI2=second service AI. S-P=serum progesterone, OCT=Open cow test based on lack of detection of interferon-tau (IFNT). P=Pregnant. NP=Open. GnRH=gonadotropin releasing hormone. S-P=Serum Progesterone, S-PSPB=Serum protein specific B. US=Ultrasound. In conventional dairies (B), when OCT accuracy is 95% or more in identifying open cows, then PGF in combination with GnRH is used to synchronize estrous after day 18 (i.e., double OvSynch or Presynch-Ovsynch). Double Ovsynch entails two ovsynch protocols, the first one is the pre-synchronization to the second ovysynch protocol. Starting around day 50 DIM, cows receive two GnRH injections, 7 days before and 3 days after a PGF treatment (GnRH-7 days-PGF-3 days-GnRH). Seven days after the last GnRH, cows receive the first GnRH of the ovsynch56 followed by two injections of PGF, 7 and 8 days later. The second GnRH injection is given 56 hours after the first PGF treatment and cow are inseminated 16-20 hours after the second GnRH treatment. Double ovsynch protocol is also a strategy to induce cyclicity in anovular cows with two consecutive ovsynch protocol that involves four GnRH injections.

FIG. 12 shows detection of IFNT concentrations and accuracy of the open cow test on days 13-31 of pregnancy. Accuracy data are based on designation of open cows based on lack of detection of IFNT on days indicated compared to ultrasound results on day 32. There were very high false negative results and very low accuracy in identifying pregnant cows in this study.

FIG. 13 shows improved detection of IFNT in both external cervical os and internal cervical swab samples using a new swab device. Accuracy data in Tables 7 and 8 revealed that both methods of collection with the new swab device resulted in a 5% false negative and 97% negative predictive value. Also when evaluating only first service cattle, the false negative cows were reduced to 3% (Table 9). The external swab samples were as accurate as the internal cervical swab samples.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a” “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal but can also be accomplished by using previously isolated cells (e.g., isolated by another person, at another time, and/or for another purpose), or by performing the methods as disclosed herein in vivo. Archival tissues, such as those having treatment or outcome history can also be used. In some embodiments, the sample is a genital tract sample.

A “control” is an alternative subject or sample used in an experiment for comparison purposes. A control can be “positive” or “negative.”

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100%, or more, increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “reduced,” “reduce,” “reduction,” or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The term “polypeptide” refers to a compound made up of a single chain of D- or L-amino acids or a mixture of D- and L-amino acids joined by peptide bonds. The terms “peptide,” “protein,” and “polypeptide” are used interchangeably to refer to a natural or synthetic molecule comprising two or more amino acids linked by the carboxyl group of one amino acid to the alpha amino group of another.

In the present disclosure, “specific for” and “specificity” mean selective binding. Accordingly, an antibody that is specific for one antigen selectively binds that antigen and not other antigens or not other antigens lacking epitope look-alikes.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd).

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with IFNT such that IFNT is inhibited from interacting with IFNT. The antibodies can be tested for their desired activity using the in vitro assays described herein, or by analogous methods, after which the in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain IFNT binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

“Radioimmunoassay” and “RIA” refers to in vitro assay techniques in which radioactive labelled antigen is mixed with unlabeled antigen (the test sample) and allowed to bind to an antibody or antigen binding fragment thereof. Bound antigen is physically separated from unbound antigen and the amount of radioactive antigen bound to the antibody is determined. The more antigen there is in the test sample the less radioactive antigen will bind to the antibody. A competitive binding assay may also be used with non-radioactive antigen, using antigen or an analogue linked to a reporter molecule. The reporter molecule may be a fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Enzyme-linked immunosorbent assays (ELISA) are standard in the art and can be found at, for example, Ausubel, F. M. et al., (Current Protocols in Molecular Biology, Volume 2, pp. 11.2.1-11.2.22, John Wiley & Sons, Inc., 1991). ELISA typically uses an enzymatic reaction to convert substrates into products having a detectable signal (e.g., fluorescence). Each enzyme in the conjugate can convert hundreds of substrates into products, thereby amplifying the detectable signal and enhancing the sensitivity of the assay. ELISA assays are understood to include derivative and related methods, such as sandwich ELISA and microfluidic ELISA.

“Breeding” as used herein refers to known methods and techniques of inseminating or impregnating an animal to produce offspring. “Breeding” includes husbandry and mating approaches, as well as non-mating approaches such as, for example, artificial insemination, in vitro fertilization, and embryo transfer.

The term “mass spectroscopy” refers to a method of detecting polypeptides, peptides, or any protein fragments thereof, following digestion with trypsin or other proteolytic enzymes that cleave a protein into smaller fragments so that molecular weight can be determined and used to confirm identity of a target protein.

### Methods of Identifying an Open Cow

The estrous cycle in cattle is about 21 days in length and is the time between ovulations based on exhibition of sexual activity (estrus). The gold standard for early pregnancy diagnostics in cows is the use of transrectal ultrasound on day 32 following AI because of ability to detect fetal heartbeat (Hansen, Proceedings of Dairy Cattle Reproduction Conference, Suppl:93-107, 2007, Hansen, et al., Reproduction, 154:F45-F59, 2017)). A chemical blood test also is accurate on day 32 based on presence of a protein in blood called pregnancy associated glycoprotein (PAG), also called pregnancy-specific protein B (PSPB) (Sasser, et al., Biol Reprod, 35:936-42, 1986). After identifying open cows on day 32 by either test, it is necessary to treat cows with hormones (prostaglandins, progestins and gonadotropin releasing hormone) so that ovulation is synchronized in these cows for AI at the best time to achieve fertilization. Consequently, these cows are not exposed to semen via second AI until day 42 following the first AI. Likewise, on organic dairy farms where estrous synchronization drugs are not used, cows must be monitored for estrus and must wait until the next natural ovulation and associated estrous response to be inseminated by AI, which does not occur until about day 42 on average (21 days plus 21 days).

The present disclosure provides methods of identifying an open cow. As used herein, an “open cow” refers to a nonpregnant cow, including ones that do not achieve pregnancy after at least one round of breeding or artificial insemination. Thus, the present disclosure provide one of skill in the art an early and efficient method of determining whether a female cow should be subjected to another round of breeding or artificial insemination.

In some aspects, disclosed herein is a method of determining pregnancy and identifying open cows, comprising breeding a cow, obtaining a genital tract sample from the cow, detecting a level of an interferon tau (IFNT) polypeptide in the genital tract sample; identifying the cow as pregnant if the level of IFNT is higher than a reference control or as not pregnant if the level of IFNT is the same as or lower than the reference control, and performing additional breeding of the cow if the cow is determined to be not pregnant.

In some aspects, disclosed herein is a method of discriminating between a pregnant cow and an open cow, the method comprising breeding a cow, obtaining a genital tract sample from the cow, measuring a level of an interferon tau (IFNT) polypeptide in the genital tract sample, detecting a high IFNT level in the pregnant cow relative to a reference control, and detecting a low IFNT level in the open cow relative to a reference control, wherein the open cow is subjected to an additional round of breeding.

In some embodiments, the genital tract sample is obtained from the cow on or before day 18 post breeding. In some embodiments, the genital tract specimen is obtained from the open cow on day 15, 16, 17, or 18 post breeding. In some embodiments, the genital tract sample comprises a swab or tissue collection from the vagina, the uterine body, a uterine horn, the cervix, an ovary, or a combination thereof.

In some embodiments, the method identifies the open cow on or before day 18 post breeding. In some embodiments, the methods identifies the open cow on day 15, 16, 17, or 18 post breeding. In some embodiments, the method detects the level of the IFNT polypeptide on or before day 18 post breeding. In some embodiments, the method detects the level of the IFNT polypeptide on day 15, 16, 17, or 18 post breeding.

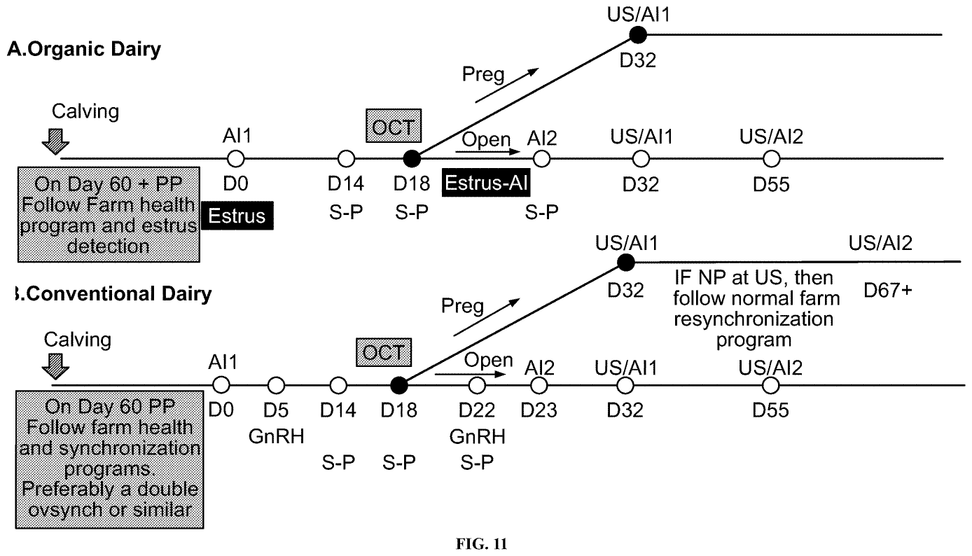

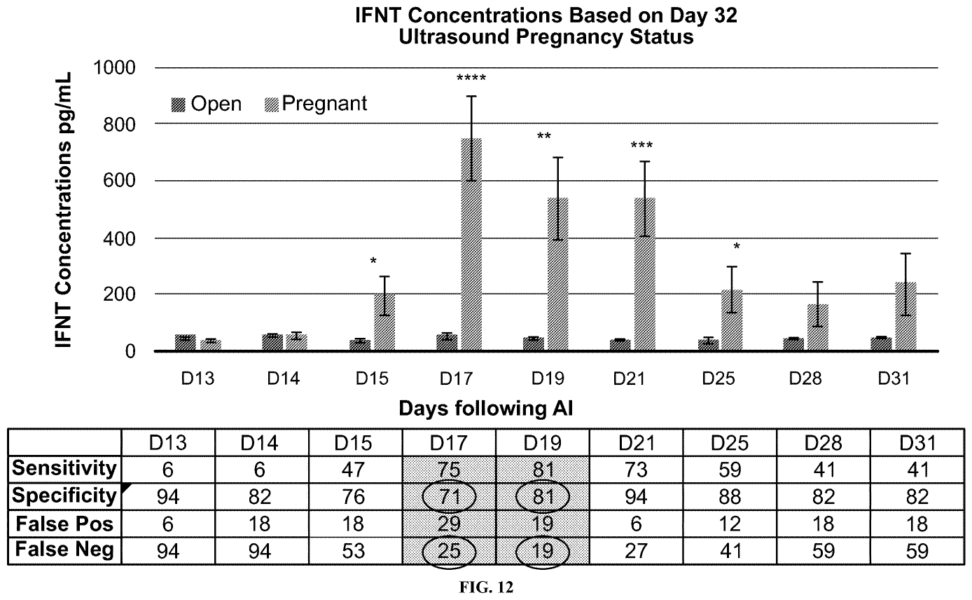

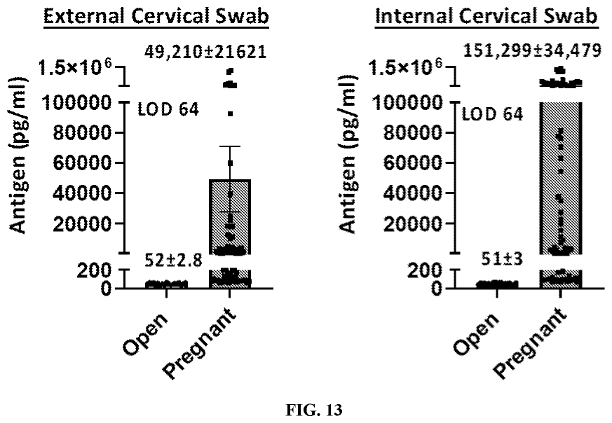

In some embodiments, the level of the IFNT polypeptide is detected by an immunoassay. As used herein, an “immunoassay” refers to a biochemical test that measures the presence and/or concentration of a biomolecule (such as IFNT polypeptides) through contact with an antibody and a detection probe/enzyme including, but not limited to horseradish peroxidase (HRP), alkaline phosphatase (AP), glucose oxidase, beta-galactosidase, luciferase, beta-lactamase, urease, a radioisotope, and a fluorophore. In some embodiments, the immunoassay comprises an enzyme-linked immunoassay (ELISA), lateral flow immunosorbent assay, radioimmunoassay, or a derivative thereof. In some embodiments, the immunoassay is performed by contacting the genital tract specimen with an antibody targeting a glycosylated IFNT polypeptide. In some embodiments, the antibody comprises a polyclonal antibody or a monoclonal antibody targeting the glycosylated IFNT polypeptide. In some embodiments, the method of any preceding aspect comprises an immunoassay comprising a cow-side lateral flow test (see FIG. 10).

In some embodiments, the glycosylated IFNT polypeptide comprises a bovine IFNT polypeptide. In some embodiments, the glycosylated IFNT polypeptide comprises a cow IFNT. In some embodiments, the glycosylated IFNT polypeptide comprises a glycosyl molecule bound to an amino acid of the IFNT polypeptide. In some embodiments, the amino acid is located at position 101 of the IFNT polypeptide.

In some embodiments, the method detects an IFNT polypeptide comprising at least 70% sequence identity to SEQ ID NO: 1. In some embodiments, the method detects an IFNT polypeptide comprising at least 80% sequence identity to SEQ ID NO: 1. In some embodiments, the method detects an IFNT polypeptide comprising at least 90% sequence identity to SEQ ID NO: 1. In some embodiments, the method detects an IFNT polypeptide comprising at least 95% sequence identity to SEQ ID NO: 1. In some embodiments, the method detects an IFNT polypeptide comprising at least 99% sequence identity to SEQ ID NO: 1. Specifically, the IFNT polypeptide can be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, or any amount in-between, below, or above these ranges. In some embodiments, the methods detects an IFNT polypeptide comprising SEQ ID NO: 1.

In some embodiments, the method detects an IFNT polypeptide comprising at least 70% sequence identity to SEQ ID NO: 3. In some embodiments, the method detects an IFNT polypeptide comprising at least 80% sequence identity to SEQ ID NO: 3. In some embodiments, the method detects an IFNT polypeptide comprising at least 90% sequence identity to SEQ ID NO: 3. In some embodiments, the method detects an IFNT polypeptide comprising at least 95% sequence identity to SEQ ID NO: 3. In some embodiments, the method detects an IFNT polypeptide comprising at least 99% sequence identity to SEQ ID NO: 3. Specifically, the IFNT polypeptide can be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 3, or any amount in-between, below, or above these ranges. In some embodiments, the methods detects an IFNT polypeptide comprising SEQ ID NO: 3.

In some embodiments, the method detects an IFNT polypeptide comprising at least 70% sequence identity to SEQ ID NO: 5. In some embodiments, the method detects an IFNT polypeptide comprising at least 80% sequence identity to SEQ ID NO: 5. In some embodiments, the method detects an IFNT polypeptide comprising at least 90% sequence identity to SEQ ID NO: 5. In some embodiments, the method detects an IFNT polypeptide comprising at least 95% sequence identity to SEQ ID NO: 5. In some embodiments, the method detects an IFNT polypeptide comprising at least 99% sequence identity to SEQ ID NO: 5. Specifically, the IFNT polypeptide can be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 5, or any amount in-between, below, or above these ranges. In some embodiments, the methods detects an IFNT polypeptide comprising SEQ ID NO: 5.

In some embodiments, the method detects an IFNT polypeptide comprising at least 70% sequence identity to SEQ ID NO: 7. In some embodiments, the method detects an IFNT polypeptide comprising at least 80% sequence identity to SEQ ID NO: 7. In some embodiments, the method detects an IFNT polypeptide comprising at least 90% sequence identity to SEQ ID NO: 7. In some embodiments, the method detects an IFNT polypeptide comprising at least 95% sequence identity to SEQ ID NO: 7. In some embodiments, the method detects an IFNT polypeptide comprising at least 99% sequence identity to SEQ ID NO: 7. Specifically, the IFNT polypeptide can be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7, or any amount in-between, below, or above these ranges. In some embodiments, the methods detects an IFNT polypeptide comprising SEQ ID NO: 7.

In some embodiments, the method detects an IFNT polypeptide comprising SEQ ID NO: 1, SEQ ID NO: 3, SEQ ID NO: 5, SEQ ID NO: 7, or a combination thereof.

In some embodiments, the method further comprises detecting a level of an additional RNA or polypeptide. In some embodiments, the additional RNA or polypeptide is interferon-stimulated gene 15 (ISG15) RNA or polypeptide, or any other IFNT-induced or pregnancy-induced RNA or polypeptide in the genital tract sample.

In some embodiments, the method further comprises detecting a level of progesterone in a genital tract sample, blood sample, or a milk sample.

In some aspects, disclosed herein an open cow assay for detecting levels of an IFNT polypeptide, wherein the assay comprises at least one antibody, a detection probe, and one or more buffers. In some embodiments, the at least one antibody is immobilized to a flat surface (including, but not limited to a cell culture plate, a multi-well plate (such as, for example a 96 well-plate or a 384 well plate). In some embodiments, the assay comprises a first antibody and/or a second antibody, wherein the first antibody targets the IFNT polypeptide and the second antibody targets the first antibody. In some embodiments, the assay detects IFNT in a genital tract sample. In some embodiments, the detection probe emits a detectable signal when the at least one antibody binds the IFNT polypeptide or the first antibody.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Open Cow Test (OCT)

Dairy cows in the United States of America have pregnancy rates of only ~40% on their first breeding by Artificial Insemination (AI) following calving. Most pregnancy losses are thought to be caused by embryo mortality that occurs before day 21 of pregnancy. The gold standard method for pregnancy diagnosis is using rectally guided ultrasound on day 32 or later after AI. This means that 60% of cows are open at the industry standard pregnancy determination on day 32 using ultrasound. Because of this low pregnancy rate, dairy cows often undergo 5 rounds of AI per year before becoming pregnant. The later in lactation that pregnancy occurs each year, the less milk production and financial return per cow in the herd.

Open cows are managed by many different protocols using prostaglandin F2-alpha (PGF), Gonadotropin releasing hormone (GnRH) and progestin treatments (i.e., CIDR). For example, treatment with PGF can lyse the corpus luteum (CL), causing the cow to return to estrus about three days later (Day 35) and be ready for breeding by AI. In a second method, a GnRH injection causes ovulation followed by CIDR progestin implants inserted into the vagina (i.e., CIDR) for 7 days to mimic progesterone production by the CL; then a PGF injection is given to lyse any existing CL followed by a second GnRH injection and breeding by AI about 54 h later. By identifying open cows using OCT, many estrus/ovulation synchronization approaches can be started on days 17-19 with AI on as early as day 21 post-initial breeding.

In the case where OCT may not be 95-100% accurate it is necessary to avoid injecting PGF to avoid lysing the CL in any False Negative (called open by OCT, but actually pregnant) cows, while injecting GnRH on day 20 with AI on day 21. This requires a strong and effective pre-synchronization program prior to first AI.

One approach entails PGF injection on day 40 of milk production (i.e., day 40 post-partum), followed by GnRH injection on day 43 and on day 50, followed by PGF injection on day 57 and on day 58 to completely lyse the CL, followed by GnRH in the PM on day 59 and timed AI in the AM on day 60. The OCT is completed on day 78 in milk (day 18 after first AI), with possible cow side serum progesterone determination on day 21 to identify open cows with very low progesterone (depends on the assay used but typically <1-2 ng progesterone/ml) that can be AI'd immediately. If Day 18 OCT or Day 18 OCT and serum progesterone on Day 21 following AI is 95 to 100% accurate when detecting open cows, then this scenario can be continued over the next 3 AI cycles. All cows called pregnant on day 18 need to be checked using ultrasound or PSPB/PAG on day 32 or later because of the expected embryo mortality that occurs in pregnant cows between day 17 to 32.

If OCT is 95-100% or use of OCT on day 17, 18, 19 or 20 with serum progesterone determined on day 21 is 95-100% accurate in identifying open cows, then many re-synchronization approaches using PGF, and GnRH are possible.

By using the open cow test (OCT) to identify open cows by day 17, one can breed open cows by AI on days 18, 19, 20 or 21 compared to day 35 or day 42 using industry resynchronization protocols. According to Cabrera and co-workers, open cows cost dairy producers about $47 per week. This means that using OCT on day 17, with AI on day 21 can assist in producing pregnancy 2-3 weeks earlier in lactation compared to cows managed by current methods. Getting cattle pregnant earlier in lactation can result in greater overall milk and calf production in subsequent years and contribute to the longevity of cows in the herd. The method of cervical/vaginal sample collection is through the use of a cotton swab device and placement of the tip of the swab containing cervical secretions into phosphate buffered saline or similar fluid. The sample is then either analyzed using the antibody-based ELISA or Lateral Flow Immunosorbent Assay to detect presence of IFNT. Cows with IFNT concentration (pg/ml) 2× or greater than the control (steer) serum background in the assay are considered pregnant in the assay. This ELISA-based background value of IFNT has been typically around 25-60 pg/ml.

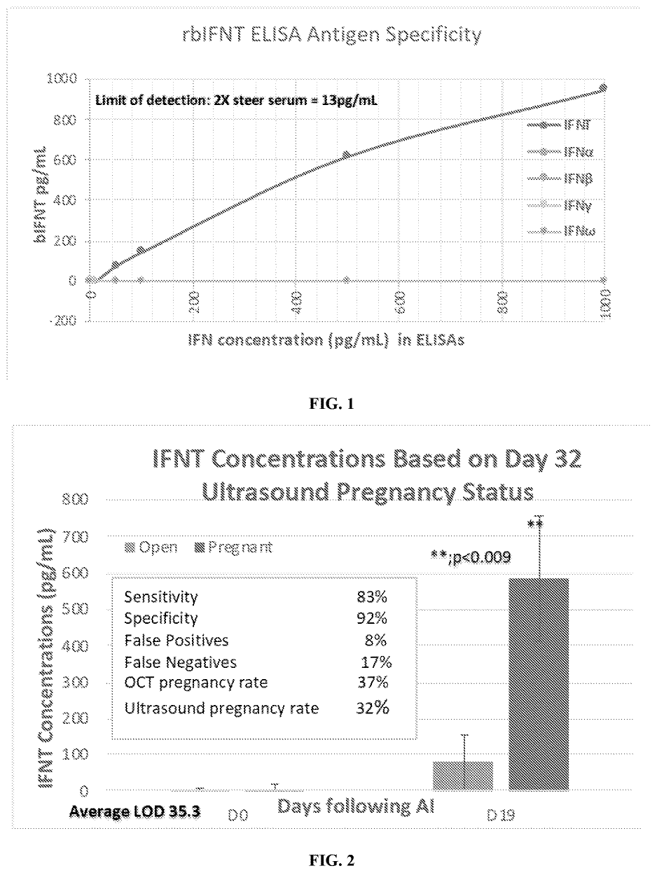

The enclosed OCT technology uses swab devices (FIG. 1) to collect cervical fluid and then it is cut with scissors directly into a sample collection tube that contains phosphate buffered saline. One versed in the artificial insemination of cows is able to use this device to collect vaginal/cervical swabs.

Identification of the open cows following AI. The bovine estrous cycle is approximately 21 days in length. Ovulation occurs on day 0 (same as ~day 21) and is followed by formation of the corpus luteum (CL) that produces progesterone. The CL typically starts to regress by day 16-17 of the estrous cycle, leading to decreased serum progesterone and ovulation by day 21 on average. For an embryo to survive, the luteolytic pathway must be suppressed or circumvented. The primary maternal recognition signal from the conceptus is IFNT, which is produced by the conceptus on ~14-20 of pregnancy. IFNT is released from the conceptus, binds endometrial receptors, and disrupts the release of PGF. By blocking PGF release, IFN-T rescues the CL such that progesterone continues to be released and the pregnancy ensues. Early (prior to day 32) pregnancy tests can result in some false-positive results due to the occurrence of 40% pregnancy losses between day 15 and 32 in cattle due to embryo mortality.

Diagnosis of pregnancy and management of the open cows is integrated into a larger reproductive management program and is justified by the excessive costs of maintaining non-pregnant (open), non-lactating cows. Early open cow diagnosis and second insemination using timed AI can reduce the number of open cows that do not return to estrus following first service and the economic consequences of delayed return to pregnancy. Diagnosis of pregnancy, or more appropriately the identification of open cows is an economically profitable venture that is dependent on several factors.

Trans-Rectal Palpation. Using palpation per rectum, pregnancy can be determined as early as 28-35 days following conception when palpating for the presence of the amniotic vesicle, fetus, placentomes, and the “membrane (placenta) slip”. The amnionic vesicle is a spherical, turgid, and fluid-filled structure that can be detected as early as 28-35 days in heifers and older cows. The placenta can also be detected as early as 30 days through gently pinching the enlarged uterine horn and then reducing pressure to allow the placenta to “slip” between the uterine walls. Detection of pregnancy at these stages through palpation per rectum using these methods is not recommended because less invasive methods such as ultrasound are much less apt to induce loss of pregnancy through mechanical manipulation of the uterine tract. Use of palpation to determine early pregnancy increases the risk for fetal damage or losses of pregnancy particularly if it involves rigorous palpation of the amnion and/or fetal membrane slip. For this reason, palpation of the pregnant uterus can be as gentle as possible and is not generally practiced until after day 39 following AI.

Ultrasound. The gold standard method for pregnancy diagnosis is using rectally guided ultrasound on day 32 after AI, which is accurate as early as day 27 of pregnancy, but also requires a skilled technician. Most breeding personnel practice ultrasound on day 32 following AI.

Progesterone. Detection of pregnancy status using serum or milk progesterone still has merit. Because most estrous cycles are 16 to 24 days in length with an average of 21 days, one can sample most non-pregnant cows at a time when progesterone concentration is low, making them distinguishable from pregnant cows with elevated progesterone concentration. The problem with serum progesterone is that it varies considerably in concentration over the 16 to 24 day estrous cycle. However, some studies showed good results: On day 21, milk progesterone levels were 97% accurate when compared to subsequent palpation data when detecting non-pregnant cows. On day 24, this accuracy decreased slightly to 95% when identifying non-pregnant cows, but the accurate detection of pregnant cows was improved (day 24 vs day 21; 88% vs 77%; respectively).

Pregnancy Specific Protein B/Pregnancy Associated Glycoprotein (PSPB/PAG). PSPB is present in binucleate cells of the trophoblast as early as day 21 of pregnancy in cows. Detection of this protein in blood is a very good indicator of pregnancy status when evaluated around day 32. PSPB is a member of the multi-gene PAG family and is identical to PAG-1. There are now twenty different PAG genes that have been identified. PSPB has an exceptionally long half-life because of the structure of the protein and extensive sugar residues (e.g., carbohydrates). Thus, it remains in circulation for several months following parturition. When cows are bred or inseminated prior to 70 days post-partum, residual PSPB concentrations reduce the accuracy of this diagnostic procedure. Regardless, use of PSPB as an indicator of pregnancy is a proven diagnostic test. Currently, blood samples are shipped to a laboratory for analysis. Implementation of an on-the-farm diagnostic might greatly facilitate further implementation of detection of PSPB as a tool to manage reproduction in dairy cows.

Implementation of the OCT ELISA or lateral-flow diagnostic test on day 18, gives a 2-week advantage in managing the open cow over this single relevant competitor, PSPB. Another option on day 32 is use of pregnancy-specific protein B also called BioPryn (biotracking.com). This product is used on day 28-32 of pregnancy. While the PSPB test works well in virgin heifers, the test is limited in cows as they need to be 60-70 days or more postpartum, because of extensive half-life of PSPB in blood resulting in residual background levels of the protein in circulation after calving. This product is currently based on blood samples which entails the use of needles, blood collection tubes, centrifuges and possible errors in animal identification numbers when transferring blood to collection tubes. Currently, there are no other competing early diagnostic tests for pregnancy in cattle. The OCT test for IFNT is a swab sample that does not require needles, vacutainers, or a centrifuge.

Interferon Stimulated Genes (ISGs). The concept of detecting ISGs in blood as indicators of pregnancy is not new, but there currently aren't any consistently accurate ISG-based protein biomarkers in blood that are useful to determine pregnancy status. However, peripheral blood mononuclear cells from pregnant sheep were found to contain greater transcription of the ISG15 and myxovirus genes. Greater levels of blood cell ISG15 mRNA were also reported in pregnant dairy cows when compared to non-pregnant cows.

Regardless of the exact mechanism of blood cell activation by pregnancy, when examining average plasma progesterone concentration and average blood ISG15 mRNA levels, both were significantly greater in pregnant cows when compared to non-pregnant dairy cows on days 17, 18, 19, and 20 and 25. Prediction of non-pregnant cows based on low blood ISG15 mRNA was 100% accurate when examining blood samples over the entire collection period. However, prediction of non-pregnant cows using only the day 18 blood samples based on low ISG15 mRNA was problematic because it was only 89% accurate in predicting cows that were not pregnant.

Additional studies focusing on cattle under different production systems revealed that ISGs increased in leukocytes in response to pregnancy on day 18 in dairy heifers, but not in lactating dairy cows. A similar study in beef cows demonstrated an increase in ISG mRNA between days 15 and 22 of pregnancy with a peak concentration on day 20. That study found that leukocyte ISG mRNA levels are a more accurate predictor of pregnancy when coupled with ultrasound determination of a CL on day 20 of pregnancy.

Blood is composed of different types of cells which include erythrocytes, immature reticulocytes, thrombocytes, granulocytes, lymphocytes, and monocytes. Variable expression of ISG15 mRNA in these cells might also contribute to variable ISG15 mRNA expression and false diagnosis of pregnancy based on high ISG15 mRNA levels. Examination of ISG15 mRNA in sub-populations of isolated peripheral blood mononuclear cells may improve the accuracy of pregnancy determination and may reduce the false negative rate when compared to examining whole blood mRNA pools. The variability in ISG15 mRNA levels in blood samples on day 18 might also be caused by a slight delay or difference in the amount of IFNT released by the conceptus due to variations in development during this period. Variations in blood ISG15 mRNA levels could also be caused by diminished IFNT release that is caused by embryo mortality.

Detection of ISGs in blood is a reasonable indicator of pregnancy status in ruminants, but improvements in methodology are needed, with more adequately defined cut-offs and easier on-farm and cost-effective methods. For example, previous studies were summarized in which PBMC ISGs were assessed for early pregnancy diagnosis and reported that the false negative results ranged from 10.1 to 17.5%. Another concern for this approach to pregnancy diagnosis in cattle is the considerable induction of ISGs by other Type I IFNs (IFNA, IFNB, IFNW) that occurs in response to viral infections and pro-inflammatory stressors. These false positive responses might be managed by testing multiple blood samples including those outside of the maternal recognition window in order to find a baseline of ISG.

Interferon Tau (IFNT). A combination of in vitro and in vivo experiments resulted in the purification of the pregnancy recognition signal IFNT and the discovery that it is a Type I IFN and the exclusive anti-luteolytic factor secreted by the ruminant conceptus. IFNT mRNA is expressed specifically by the mononuclear trophectoderm cells of the conceptus from at least day 15 to 25 with peak levels on day 17-19 of pregnancy. IFNT protein has been described in secretory products from cultured embryos representing days 16 to 25 of pregnancy. Because IFNT is expressed only in conceptus trophectoderm, it was reasoned IFNT might be present in the blood and provide an indicator of pregnancy status in cattle. Historical assays for IFNT did not have sensitivity beyond the low ng levels and were not able to detect IFNT in fluids other than embryo culture media or uterine flushing.

Other markers for pregnancy status. Recently detection of melatonin in plasma samples has been associated with pregnancy status in cattle. Likewise, a few other PBMC mRNA markers might be used to determine pregnancy status: TLR2, TLR4, STAT 3, ILIB, PTGS2, PLA2G4A and ALOX5AP. All of these markers function in other physiological systems and are not specific to pregnancy. Several other protein markers have also recently been identified for pregnancy status in both conceptus secretory proteins as well as endometrial secretory proteins. However, IFNT is an interferon that is produced only by the trophoblast cells of the developing and elongating conceptus.

### Example 2. Methods

### Bovine IFNT ELISA Methods

Generation of recombinant glycosylated bovine IFNT. The full-length 1.1 kb cDNA clone encoding IFNT, called BTP509, was isolated, subcloned into pUC13. The BTP509 clone was expressed as a recombinant protein and for generation of anti-bIFNT antibodies in sheep, goats, and mice. This Animal Pharma Company engineered a C-terminal 6× HIS tail that aided in purification and was subcloned into a protein expression vector by a pharma partner and expressed in HEK transient culture. The rbIFNT was glycosylated and retained anti-viral activity. The identity of glycosylated recombinant bIFNT protein was confirmed following expression in Hek 1 cells in western blot by using a limited stock of anti-boIFNT antibody and by mass spectroscopy determination of identity of several peptides with IFNT.

Generation of polyclonal rabbit and goat antibodies against rbIFNT. The rbIFNT was used to prepare antibodies. Several booster immunizations and bleeds from rabbits and goats were tested for ability to detect rboIFNT in western blot, radioimmunoassay, and sandwich ELISA approaches. Polyclonal antibodies were produced against rbIFNT in six rabbits and three goats by the pharma partner and a subcontractor using a Freund's complete/incomplete adjuvant protocol. Western blots of rbIFNT were performed that tested the reactivity of IFNT polyclonal antibodies in raw sera. The average titers of the primary goat sera were 1:1600 (~47 ug/mL total protein) and the rabbits, 1:16,000 (4.7 ug/mL total protein).

The rbIFNT Sandwich ELISA Protocol

Goat and rabbit polyclonal sera were purified over rProtein A/G GraviTrap equilibrated columns (SIGMA) by mixing 2 ml of sera to an equal volume of 1× binding buffer. Columns were washed with 15 mL of 1× binding buffer. Neutralizing buffer pH 9.0 (60 uL) was added to each of 10 1.5 mL elution tubes and antibodies were eluted in 0.5 mL fractions with 5 mL of 1× elution buffer. Protein concentration of the fractions was determined by BCA and positive fractions pooled. Protease inhibitor was added to the positive pool at a 1:200 dilution.

Numerous matrices of capture and detector antibodies and rbIFNT concentration were performed to determine best polyclonal antibody concentrations for the ELISA. Goat #51 PAB was plated at 50 uL/well (96-well high binding microplate #655081 Fisher Scientific) as the capture antibody at a concentration of 3.5 ug/mL in carbonate coating buffer (0.2M Sodium Carbonate/Sodium Bicarbonate pH 9.4) for 2 hour at 25° C. Plates were washed three times with 0.2 mL/well of (0.025M Tris, 0.15M sodium chloride pH 7.2, 0.05% Tween 20). The plate was blocked 21 hours at 25° C. in 0.3 mL/well 2% BSA blocking buffer (0.025 M Tris, 0.15M sodium chloride pH 7.2, 0.05% Tween 20). In order to generate a standard curve rbIFNT protein was serially diluted 2-fold in steer serum from 500 μg/mL to 7.8 pg/mL. 50 uL/well of each undiluted swab in 1×PBS sample and rbIFNT standard dilutions were added to duplicate wells and incubated for 24 1 hours at 37° C. for 1 hour. Plates were washed three times with 0.2 mL buffer/well and then 50 μL/well of detector antibody (concentration; rabbit 5670 PAB) conjugated to biotin (FluoReporter Mini-biotin-XX Protein Labeling Kit #F6347, LifeTechnologies, Eugene, OR; 9.6 pg/mL), was added into 1:5 diluted blocking buffer and incubated at 25° C., for 1 hour, in the dark. The plates were washed 3× and 50 μL/well of SA-HRP (#DY998 R&D Systems, Minn., MN) diluted 1:200 in 1:5 diluted blocking buffer was added and then the plate was incubated for 30 minutes, at 25° C., in the dark. The plates were washed 6× and 50 μL/well of an equal volume mixture of TMB A and B substrate (#42110 BioLegend, San Diego, CA) was added and the plate incubated for 8-10 minutes at 25° C. in the dark. The reaction was stopped with 50 μL/well of 1.6M H₂SO₄. The plates were read at 450 nM on a BioTech Synergy 2 reader. Steer serum collected from one steer was used as diluent for the rbIFNT standard quality controls and background. 1× PBS was used as an additional control. Quality control rbIFNT standards diluted in steer serum (500 μg/mL, 100 μg/mL, and 20 μg/mL) were thawed and 50 μL/well added in duplicate wells to each plate.

### Example 3. Open Cow Test Results

Each standard/specimen is added to duplicate wells. The OD 450 nM is averaged for each steer serum control, standard dilution, specimen, and QC control. The rbIFNT standard protein was serially diluted 2-fold in steer serum beginning with 500 μg/mL and ending with 7.8 pg/mL. Steer serum served as the negative background control. The average rbIFNT standard absorbance at OD450 nM was plotted in EXCEL to determine the rbIFNT concentrations. bIFNT concentrations in cervical swabs from individual animals were calculated from a bIFNT standard curve. The R² best fit relates how close the linear standard is to 1. Concentrations in this study were reported in pg/mL.

The Coefficient of variation (%) is calculated as the standard deviation between rbIFNT OD450 absorbance wells/means×100. In these studies, the bIFNT ELISA had an average stringent limit of detection, (LOD), down to ~53.7 pg/mL (2× the OD450 0-value) or less stringent LOD of 6.6 pg/mL (OD450 0-value+3* SD 0 value) and the limit of quantitation, (LOQ), was 7.1 pg/mL (OD450 0-value+10*SD 0-value). The Student T test was used to identify statistically significant differences between “open” cows and pregnant.

The IFNT ELISA was tested for antigen specificity to bIFNT. Other type 1 IFN proteins such as IFNA, IFNB, IFNW and IFNG were tested on the same plate as rbIFNT with protein concentrations of 1000 pg/mL, 500 pg/mL, 100 pg/mL, 50 pg/mL, 10 pg/mL, 5 pg/mL, and 1 pg/mL and did not interact with the anti-rbIFNT protein antibody.

Accuracy of the bIFNT ELISA to identify open cows compared to ultrasound diagnosis on day 32. The following open cow test (OCT) assay features compared to day 32 ultrasound were calculated for each assay and each day following AI tested. Note that pregnant cows are considered positive in this example. A high False Positive (and low specificity) can be seen because embryos that are present often (~50%) die prior to ultrasound on day 32. However, this technology is not describing a pregnancy test. Rather the open cow test (OCT) focuses on identifying the open cows so that they can be better managed. The two factors that are of concern for this test are the False Negative and Specificity estimates. Considering the False Negative group, it must be low to avoid aborting any pregnant cows if rebreeding strategies are using PGF2α. If the False Negative value is significant >10%, then this test is still useful, but strategies for rebreeding that do not include PGF2α are described (see Rebreeding Strategies).
Sensitivity: Correct ELISA pregnant=# Pregnant by both ELISA & US/# pregnant by ultrasound
Specificity: Correct ELISA open=# Open by both ELISA & US/# open by ultrasound
False Positive: ELISA called pregnant when actually found to be open by ultrasound (# ELISA false pregnant/# open by ultrasound)
False Negative: ELISA called open when actually found to be pregnant by ultrasound (# ELISA false open/# pregnant by ultrasound)
   Note that the OCT ELISA detected only bIFNT and did not cross react with the other IFN family members (FIG. 1).

Serum, Plasma and Milk testing for IFNT using the IFNT ELISA. After a couple of years of testing blood and milk samples for bIFNT using ELISA, it was realized that even with a limit of detection of 30-50 pg/ml, IFNT may not be consistently detected in these fluids from day 15-21 pregnant cows. There can be methods to enrich for IFNT in these samples, but other simpler methods were considered first. Other bovine fluids are tested. It was reasoned that in lactating cows, the cervix was dilated during and for a time after calving. There is no formal “plug” that forms in the cervix following fertilization. Because IFNT has been reported to be produced in levels as high as 500 microgram/24 h from bovine conceptuses cultured in vitro, it was reasoned that enough IFNT can escape the uterus and be detectable in vaginal/external os cervical swabs. By using a double-guarded uterine swab device, vaginal/external os cervical fluid is swabbed on specified days following AI and then placed the tip of the swab into a small Eppendorf tube with 0.5 ml phosphate buffered saline. Samples were then taken back to the lab and analyzed in ELISA which takes about 30 h from start to finish to complete. The primary advantage of the cervical swab compared to blood samples is that there is no needle or vacutainer necessary and there is no need to centrifuge blood samples to yield plasma or serum.

Performance of Open Cow Test (OCT) when Identifying Open Cows in Vaginal/Cervical Swabs

Animals—The cooperating dairy producers are located near Mead, Colorado (Dairy A) and provided the experimental lactating dairy cows. Holstein, Jersey, and Brown Swiss cows (112 total cows) were entered in this protocol, 19 in experiment #1 (9 Holsteins, 9 Jerseys and 1 Brown Swiss); 36 in experiment #2 (15 Holsteins and 21 jerseys), respectively; 20 in experiment #3 (11 Holsteins, 8 Jerseys and 1 Brown Swiss), respectively and 37 in experiment #4 (17 Holsteins, 19 Jerseys and 1 Brown Swiss), respectively. All animal experiments and procedures were approved by the Institutional Animal Care and Use Committee at Colorado State University.

Synchronization protocol—Dairy A—A modification of the G6G protocol was used to synchronized estrous. Cows received 2 ml of cloprostenol sodium (prostaglandin F2α analogue; Estrumate, Merck) i.m. on day 39 and 53 post-freshening to induce luteolysis of all mid- and late-cycle corpora lutea. Two days later (day 55), cows received 2 ml of GnRH (gonadorelin, Merck) i.m. to induce ovulation. Six days after the first GnRH injection (day 61), cows received another 2 ml of GnRH. Six days later (day 67), cows received 2 ml of Cloprostenol sodium to lyse any CL followed 7 days later by 2 ml of GnRH. Cows were inseminated 16 hours later. Cows were pregnancy checked by transrectal ultrasound at days 32, 46 and 60 and palpated at days 110 and 180.

Vulva Swab—The vulva and immediate area was cleared of manure with a paper towel. After collection, the swab was placed in a 5 mL tube containing 1 mL of 1×PBS and placed at 4° C. blue ice blocks for short-term storage. Upon return to the lab the tubes were vortexed for 5 seconds and the swab removed with sterile forceps. The tubes were stored at −20° C. until all samples had been collected and the ELISA was performed.

Cervical Swab—The sterile culture swab enclosed in a retractable sheath (Jorgensen Laboratories, Inc) was inserted deep into the vagina avoiding the urethra and contacting the deep vagina and/or external os of the cervix. The sheath was retracted exposing the sterile swab, rotated several times then drawn back into the sheath, the depth of the swab was noted and removed from the vagina. The swab was placed in a 5 mL tube with 1 mL of 1×PBS and held at 4° C. on blue ice. Upon return to the lab the tubes were vortexed for 5 seconds and the swab removed with sterile forceps. The tubes were stored at −20° C. until all samples had been collected and until the ELISA was performed.

Data—Means were considered different if P<0.05. Data are presented as means±standard error. were analyzed on each day using protected T-tests comparing levels of IFNT in open compared to pregnant cows based on ultrasound at day 32. Open cows were identified by IFNT values<2× the background values of steer serum controls (limit of detection; LOD) controls on each ELISA plate.

Use of OCT to Identify Open Cows and Comparison with Serum Progesterone and PSBP/PAG

Blind vaginal swab of cows 19 days after AI in 19 cows. Vaginal swabs were collected blindly by inserting the swab device into the vulva and as deep as possible into the vagina and then twirling the swab device for about 30 seconds as deep as possible. The overall pregnancy rate using ultrasound was 37%. Using the ELISA, all cows with IFNT concentrations greater than the 35.3 pg/ml Limit of Detection (LOD) were called pregnant and all cows with values equal to or less than 35.3 pg/ml were called open. LOD is defined as 2× background values for steer serum.

Identification of open cows on day 19 using IFNT concentrations the LOD for the assay were provided to the dairy before the dairy disclosed their day 32 ultrasound data. Note Negative Predictive Value and Specificity of 92% when correctly identifying open cows. There was only an 8% error rate when identifying open cows; although, there was a 17% false negative rate (falsely calling a pregnant cow open). This experiment was the first time this analysis was completed, showing good results with an unrefined blind vaginal swab collection. It was then decided to determine the most accurate day for determining pregnancy status based on IFNT swab sample concentrations of IFNT.

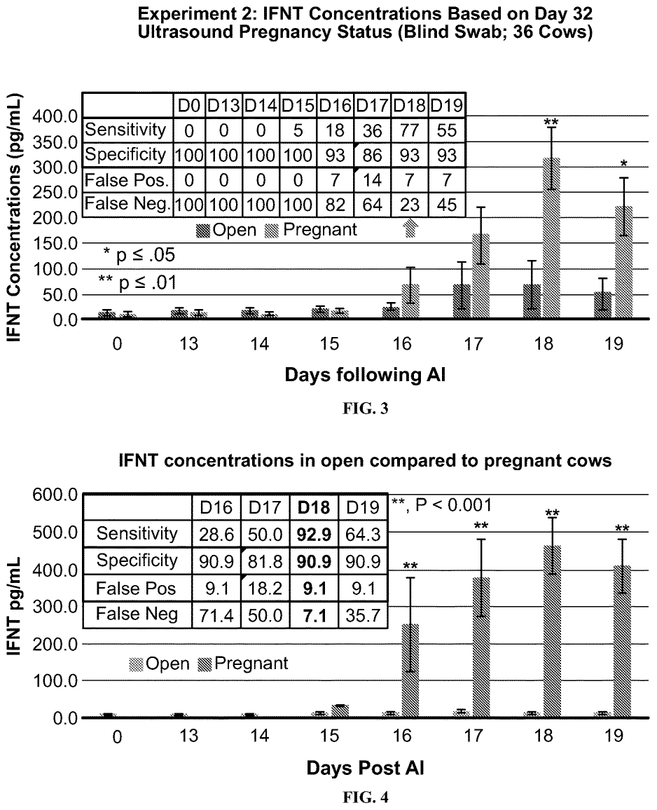

Determination of the best (most accurate overall) day for identifying open cows following AI based on limit of detection of (LOD) IFNT concentrations (FIG. 3). Blind vaginal swabs were collected on day 0, and on days 13-19 following AI and analyzed for IFNT concentrations using the IFNT ELISA. IFNT was only significantly increased in pregnant cows on day 18 and 19 following AI. The most accurate day of predicating open cow status was on day 18 in context of 93% specificity and 23% false negative rate.

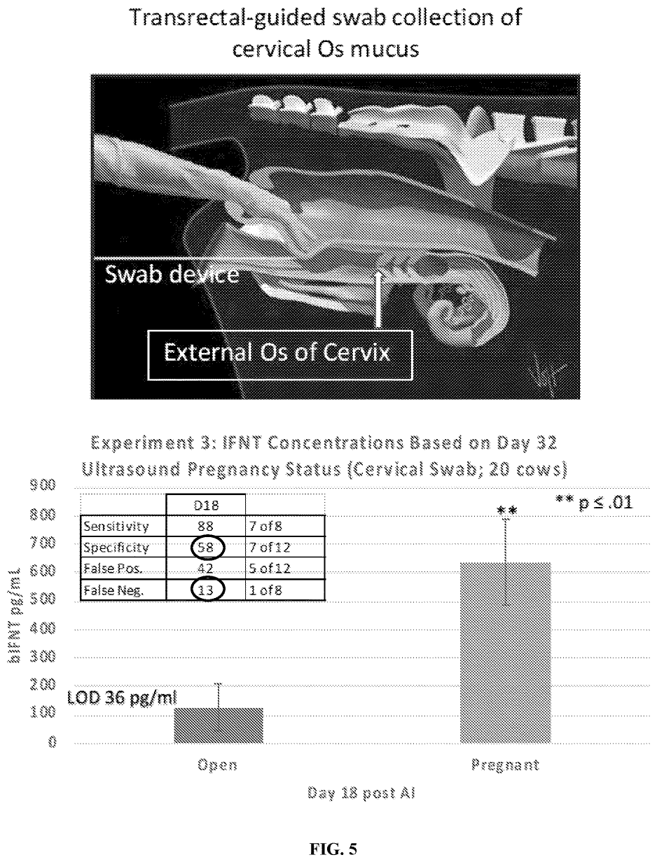

The inability to detect IFNT in 23% of all pregnant cows in this study (False negative rate for day 18 in FIG. 3 was disappointing. The data in FIG. 3 included blind vaginal swab samples from first calf heifers and from lactating cows. A subset of these data representing only the lactating dairy cows (data with first calf heifers removed) is shown in FIG. 4. The results are more accurate in cows that had calved and had cervices that were dilated two or more times compared to young heifers with only one calving and cervical dilation prior to swab collections. Notice sensitivity and specificity in the 90%+ range and only 9.1% false positive and 7.1% false negative results in this set of lactating cows only with first calf heifers removed. It was contemplated that failure to detect IFNT in some pregnant cows in FIG. 4 is caused by blind sampling of the vagina in a location that was not in close proximity to the cervix, the source of IFNT release from the uterus. To test this idea, the swabs were guided to an area near the external os of the cervix using transrectal palpation similar to the AI technique, without entering the cervix with the sampling device on day 18 following AI (See FIG. 5 upper panel).

IFNT concentrations in cervical/vaginal swab samples on day 18 in open and pregnant cows based on retrospective ultrasound on day 32 (FIG. 5 lower panel). IFNT concentrations were greater in pregnancy compared to open cows in cervical/vaginal swab samples on day 18. Although accuracy of the test in context of specificity actually decreased and false positive rate increased to 42%. The false negative rate improved to 13%. It was not clear why these day 18 guided swab results were so poor and in light of the smaller 20 cow sample size, and thus it was decided to repeat the time course study using more cows and this more directed swab collection to the cervical os area in the reproductive tract.

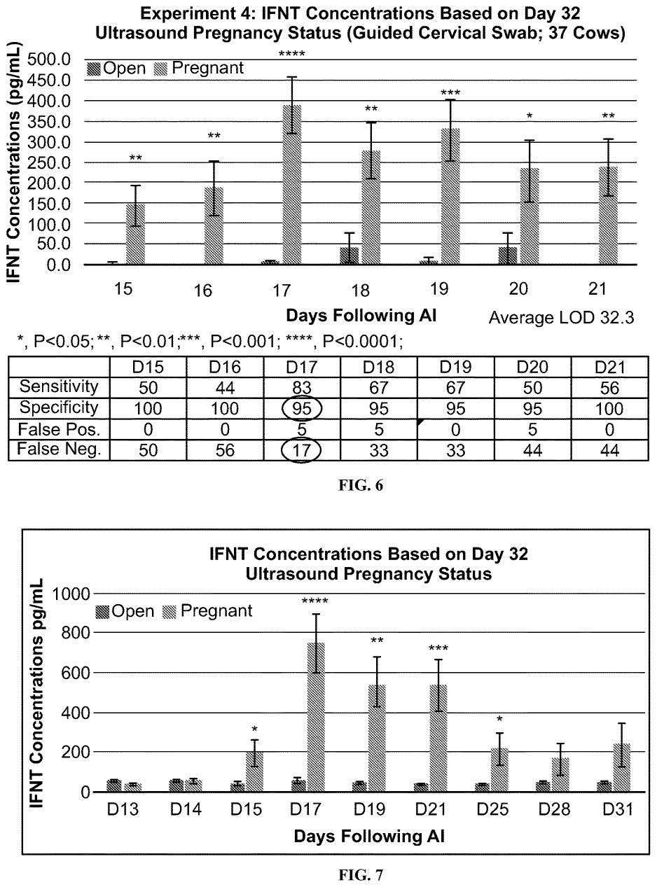

Time course of IFNT concentrations in trans-rectal guided swab collections of cervical os fluids in open and pregnant cows. Shown in FIG. 6 is detection of IFNT in guided cervical swab samples by day 15 with continued detection through day 21 in pregnant compared to open cows. The best accuracy values were on day 17 following AI: 83% Sensitivity, 5% False Positive Rate, 95% Specificity and 17% False Negative Rate.

Notice in all open cows that background IFNT concentrations were less than the 32.3 pg/ml LOD for this assay and in all pregnant cows the IFNT concentrations were greater than the LOD. In cows that lost embryos because of early embryonic mortality (EM), IFNT concentrations were higher than the LOD for one or two days, but then decreased to background LOD. Profiling IFNT concentrations from days 15 through 21 can be used to identify cows with EEM. IFNT in swab samples after day 21 had not been measured yet, but these times can be examined to determine which pregnancies are failing because of EM (also see FIG. 7). Pregnant cattle consistently had IFNT concentrations above the LOD. Some pregnant cows consistently had low IFNT concentrations in swab samples. On day 17, the 17% false negative rate reflects this problem with the assay. The lack of detection of IFNT in the false negative cows was consistent across all days of swab collections. These cows can be studied in the context of the reproductive anatomy to understand why IFNT was not detected in the guided external cervical swab samples. Perhaps the uterus is so large in some cows that it pulls the reproductive tract downward limiting the amount of uterine fluid leaking out of the cervix. In these cases, elevating the uterus just prior to sample collections reduce these false negative cows. Likewise, inserting the swab into the cervix about 1 cm also improves detection of pregnant cows (Sensitivity) and reduce the false negative rate for calling cows open when in fact they are pregnant.

More expanded analysis of IFNT levels following AI. A more expanded time course of cervical swab collections was completed to confirm the best day following AI for testing absence of IFNT in open cows (FIG. 7). Swabs were collected from days 13 through 31. Note that the best False Negative results occurred on Day 19 at 19%. Days 17 and 21 also had 25% and 27% False Negative results as well. Day 17 was identified as the best day to collect samples in the previous time course and it was fairly close to the Day 19 results in this study. Also, Day 17 worked best at the cooperating dairies in context of their management systems. For this reason, clinical trials of ~330 cows were set up at two cooperating dairies for cervical swab collections on Day 17 following AI. Note that all of the previous studies were completed by the Hansen Laboratory (Technician 1) on Dairy 1. Data in FIG. 7 were collected by a second technician

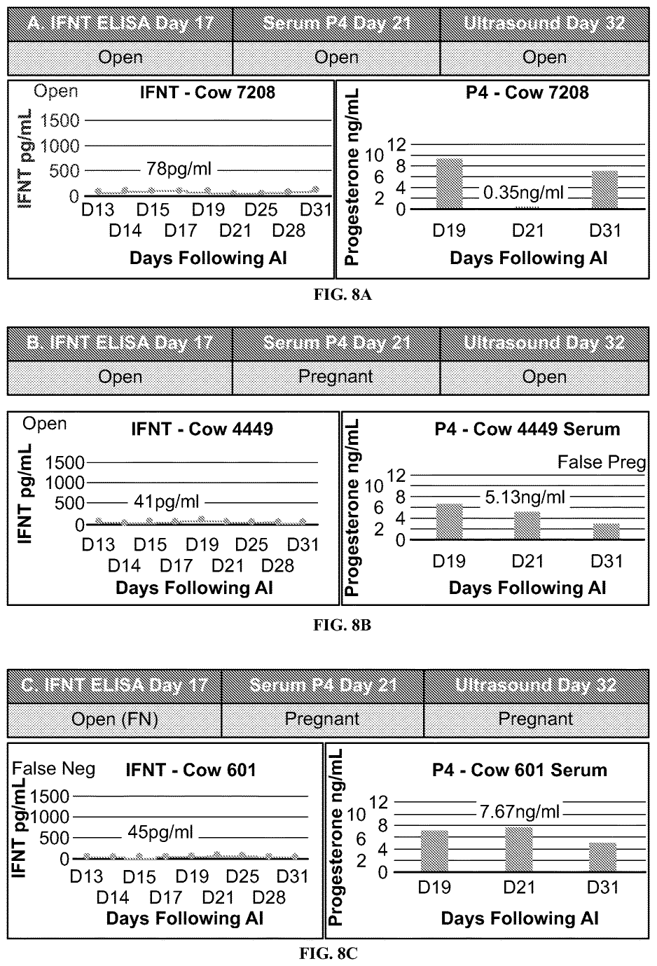

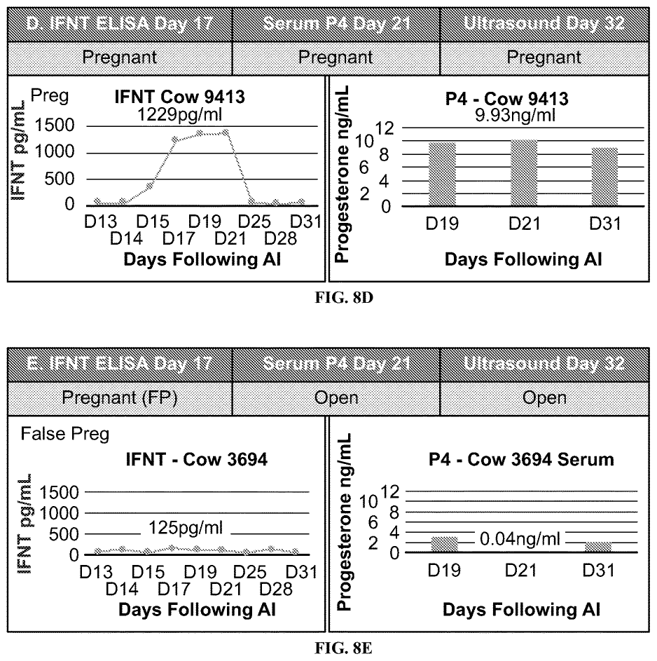

Comparison of OCT IFNT levels on Day 17 and serum P4 levels on Day 21. By day 19, serum progesterone concentrations trend down a bit in open cows reflecting the start of luteolysis. Serum progesterone concentrations are lower in open compared to pregnant cows on day 21. The serum progesterone concentrations on day 31 were determined to see if they correlated with the ultrasound call for pregnancy. Notice in cows that were called open by ultrasound, OCT, and serum progesterone (FIG. 8A), that IFNT levels were low at 15-36 pg/ml which is essentially background in the assay because open cows do not produce IFNT. This was coupled with very low serum progesterone: 0.04 to 1.1 ng/ml. In the false positive cows determined by OCT (FIG. 8B), IFNT levels ranged from 78 pg/ml to 125 pg/ml, which is at the low end for pregnant cows, but serum P4 ranged from 0.04 to 0.82 ng/ml and was clearly indicative of open cows. In this case use, of both serum P4 and OCT to make an open cow determination would have resulted in 0 false positive cows. In false positive cows based on serum P4 only (FIG. 8C), serum progesterone ranged from 1.5 to 5.13 ng/ml and IFNT ranged from 20-52 pg/ml. Depending on the cut off for serum P4, whether it is 1 ng/ml or 2 ng/ml, this false positive rate would be reduced at the higher cut off. Also, by using both serum P4 and IFNT, these false positive cows would be suspect in context of pregnancy status. One false positive cow out of the group tested was called pregnant by OCT and P4 on day 17 but was found to be open on Day 32 based on ultrasound. Clearly many of the OCT and serum P4 false positive cows had embryos that died prior to day 32 ultrasound.

In cows called open based on OCT, serum progesterone and ultrasound (FIG. 11A), IFNT levels ranged from 70 pg/ml to 1451 pg/ml and progesterone levels ranged from 3.6 to 14.6 ng/ml. In cows called open by OCT when in fact they were pregnant by ultrasound, IFNT levels ranged from 26 pg/ml to 53 pg/ml and serum P4 ranged from 5.6 to 14.6 pg/ml. These false negative cows identified on day 17 would have been identified using serum progesterone as a secondary marker on day 21. Note that there were no pregnant cows based on ultrasound and OCT that were open by P4. Likewise, there were no pregnant cows by ultrasound that were called open by OCT and P4.

Clinical trials comparing OCT to ultrasound. The first clinical trial entailed sampling 332 lactating dairy cows on day 17 following AI at a dairy located northeast of Fort Collins, CO (Dairy 2). These samples were collected by Tech 2. The accuracy estimates are listed in Table 1. There was a 35% pregnancy rate based on ultrasound on day 32. The specificity was 82% and the false negative rate was 21 percent at this location.

Table 1 shows the Accuracy of Open Cow Test (OCT) when identifying pregnancy status on Dairy 1 (Tech 3) in Colorado. Sensitivity: ELISA pregnant=# Pregnant by both ELISA & US/# pregnant by US. Specificity: ELISA open=# Open by both ELISA & US/# open by US. False Positive: ELISA called pregnant when it was called open by ultrasound (# ELISA false pregnant/# open by ultrasound). False Negative: ELISA called open when it was call pregnant by ultrasound (# ELISA false open/# pregnant by ultrasound).

The second clinical trial was completed on 309 lactating dairy cows at Dairy 1 located south of Fort Collins. The pregnancy rate was 49% based on ultrasound. Specificity was 88% and False Negative rate was 28% when using day 17 OCT to identify open cows.

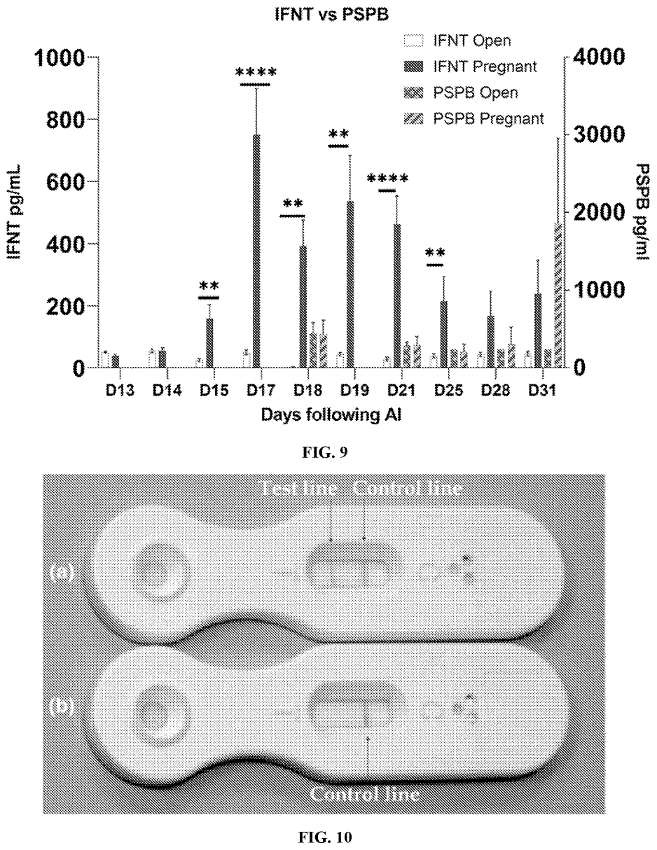

Description of temporal detection of IFNT compared to PSPB. IFNT is generally detected in cervical swab solutions between days 15-25 following AI. PSPB is generally detected in whole blood (serum) between days 25 and 32 following AI. Cervical swab solutions were submitted from two separate time course experiments to BioTracking for PSPB detection. The bIFNT values determined by OCT and PSPB values determined by BioTracking were overlaid in FIG. 9. Note that PSPB was not significantly detected in swab samples, although it appeared to start increasing by day 31 following AI.

Example of profiling embryo mortality. Profiling embryo mortality in cattle based on IFNT concentrations in cervical swab samples may be used as a bioassay for testing side effects of drugs, vaccines, insecticides, antibiotics, anti-inflammatories, or other treatments prior to or following pregnancy in ruminants

Improvements in the IFNT ELISA. More recently, the turnaround time for completion of the assay has been improved from 30 h to 5.5 h from start to finish in the laboratory. A rapid cow-side test for IFNT also is being developed in the laboratory.

In order to shorten the duration of the OCT bIFNT ELISA the following changes were made. Goat 51 capture antibody and 2% BSA blocking steps were incubated for 1 hour at room temperature and the antigens were incubated for 1 hour at 37° C. All IFNT standards and cervical swab solutions were diluted in 10% steer serum. For example: 12 μL of steer serum was added to 108 μL of cervical swab solution, mixed then 50 uL in duplicate was added to ELISA plate wells. Diluting rbIFNT in 10% steer serum for standards elevated the limit of detection about 2-fold (from 35 mL to about 70 pg/mL). Raising the LOD enabled us to call “open” a portion of samples that would have been inaccurately called positive by OCT.

Product/Service Provided: 5.5 h ELISA vs Cow-Side Lateral Flow Device. With a 5.5 h turnaround time, one can receive samples from a dairy by 8 AM and have the results back to the dairy electronically by 1:30 PM so that the dairy producer can make a decision on how to manage the open cow on the same day as the swab collection. A second product or service is a point-of-need lateral flow device. A plastic cartridge can be sent with buffer solution along with the swab device so that swab samples can be collected. There is a collection tube containing a buffer to help with transfer of the sample on the cotton swab to solution. A specific volume of this sample is placed on the lateral flow device that has primary antibody affixed to the paper matrix. A buffer containing the second antibody and detecting reagent is used to develop a signal. Presence of a “test” signal represents a pregnant cow. Absence of a signal indicates an open cow.

“Lateral flow immunoassays” (LFIAs) are a good platform for point-of-care testing when the antibody reagents are known to be highly specific to a unique antigenic biomarker. The human home pregnancy test and influenza A/B tests are ubiquitous examples of the value of the LFIA platform. The ideal cow side LFIA device is rapid (<20 min), inexpensive (~$5), and exhibits extremely high diagnostic specificity, that is, high rate of true negatives. LFIAs use a series of porous membranes to wick sample across a detection zone, as shown in FIG. 10. When a sample is added to one end of the devices, capillary forces in different porous structures transport the liquid to the other end. A waste pad ensures that the entire sample flows over the test and control lines printed on the nitrocellulose membrane. Secondary antibodies are conjugated onto gold nanoparticles (AuNPs) and dried onto a separate pad. If the sample contains the antigenic target biomarker, the conjugated antibodies bind to the target and the complex flows through the nitrocellulose membrane. The test line contains capture antibody that immobilizes the AuNP conjugates if target is present. The positive control line consists of recombinant target and captures the AuNP conjugate whether or not the target is present in order to confirm correct device performance.

The target is bovine interferon tau (bIFNT), and the capture and secondary antibodies are polyclonal goat and rabbit Abs. A 3×15 mm2 nitrocellulose membrane (GE FF120) striped with capture antibody (test) and antigen (control) is connected to a waste pad made of GE CF4 membrane. The antibody and recombinant antigen is striped onto the nitrocellulose strip with a reagent dispenser (Claremont Bio). The striping solution contains 45 mM trehalose, 4.5% glycerol, and 0.5 to 2.5 mg/mL protein. The trehalose and glycerol are used to improve storage capability. Approximately 100 to 500 ng of each protein is added to the 3 mm nitrocellulose strip. The secondary antibody is conjugated to 40 or 80 nm diameter AuNPs (nanoComposix). The conjugate is diluted in a solution of 0.01 M FeSO4-EDTA, 4% Trehalose, and 0.1% BSA to improve long term storage. Two 5 μL aliquots of the secondary antibody solution (50 ng) are sequentially dried onto the 3×5 mm2 conjugation pad.

### Description of Double Antigen Tests for Open Cows.

### Double Detection Methods (Days 17, 18, or 19: Days 17-19).

IFNT and ISG15 on days 17-19
IFNT on day 17-19 with Progesterone on day 21 or morning of AI (Cow side)
IFNT on days 17-19 with Estradiol on day 21 or morning of AI (Cow side)
IFNT on days 17-19 and PSPB/PAG on days 27-32
IFNT on days 17-19 and Melatonin on days 17-19

### Estrous Synchronization Methods on Day 17-19.

These approaches can be adjusted based on day of detection of IFN using OCT on day 17, 18 or 19 following AI.

Organic Dairy. No hormones or drugs are used on organic dairies. The approach on the organic dairies is to identify the open cows by OCT and then separate them from the pregnant cows so that they have a smaller group of cows to observe for estrus and AI or monitor for activity using pedometers and AI. Some of these dairies might elect to AI on day 20 and 21 based on the OCT.

Conventional Dairy. The protocols in the Beef Cow Protocols (Beef Reproduction Task Force) as well as other published strategies for synchronizing cattle can also be implemented. Where OCT has a false negative rate greater than 10%, then a PGF injection to lyse the CL is not indicated. Instead, producers may follow the suggested protocol below after identifying open cows using OCT on day 18. In this case producers can identify open cows on day 18 and then follow the organic dairy protocol. Or they can identify the open cows on day 18 and then treat with GnRH on day 18, 20, or 21 to induce ovulation for AI about 12 h after the GnRH injection. The best day to give GnRH is preferred on day 20 followed by AI on day 21. One other approach is to insert a progesterone releasing device called CIDR into the vagina on day 12 to day 19, with OCT on day 19, GnRH on day 20, and AI on Day 21. This can actually bring some cows into heat (ovulation) that may not have ovulated at the first AI.

If a double antigen test for IFNT on day 17-19 and serum or milk P4 on day 21 is used, then the best cows for AI are identified. Based on data in FIGS. 7 and 8, use of both IFNT and serum P4 reduces the False Negative and False positive rates. A change in threshold of IFT LOD may also reduce False Positive Rates, however the false negative rates are the most important to consider on days 17-19 following AI. A comparison of savings in expenses when maintaining open cows ($47 per week) by using OCT vs conventional ultrasound is described in FIG. 11.

In the case of 95-100% accuracy determining open cows on days 17-19, a protocol is described. This protocol uses PGF (PG), GnRH (GN), OCT and timed AI (TAI) in FIG. 11 across 4 inseminations starting on day 40 postpartum (days in milk). This system describes a short PreSynch of 7 days followed by a 7 day Ovsynch program. At the beginning of the Ovsynch protocol, all cows have a CL. For example, cows presented for first AI service have a CL prior to starting the Ovsynch and cows not pregnant go through a rebreeding sequence of PreSynch-Ovsynch for 2^{nd} service and then have a CL at the beginning of the Ovsynch component. This can be replicated four times demonstrating the sequential management through timely use of the OCT procedure of early pregnancy diagnosis at 17-19 days following AI. This protocol restores or recapitulates the ovary-uterus and induces a fresh dominant follicle for timed AI.

### Benefits of the Technology

1. Describes development of an operational enzyme linked immunosorbent assay (ELISA) and a lateral flow immunosorbent assay using antibodies against IFNT for the purpose of identifying the open (not pregnant) cow earlier than day 18.
2. Describes uniquely identifying open cows on day 15-21 following first breeding with artificial insemination (AI) and reproductive management by using estrous synchronization hormones to resynchronize and artificially inseminate open cows by day 21 of the estrous cycle.
3. Describes multiplexing low IFNT concentrations on day 19-21 with low serum progesterone concentrations to confirm open cow status.
4. Describes multiplexing low IFNT and low ISG15 concentrations on day 15-21 to identify open cows.
5. Describes tracking IFNT concentrations to identify cows with embryo mortality.
6. Describes using detection of IFNT as a bioassay for embryo health when testing if a pharmaceutical or drug treatment causes embryo mortality. For example, detection of IFNT can be used to determine if a vaccine, drug, or other treatment induces a greater risk of embryo mortality.
7. Generally, describes modification of a cervical swab device to also include a cow-side lateral flow immuno-assay point of need assay.
8. Demonstrates that detection of IFNT can be used 2 weeks earlier than reliable detection of Pregnancy Specific Protein B (PSPB/PAG) or ultrasound on day 31-32.
9. Outlines double antigen detection assays to identify open cows.
10. Describes re-breeding approaches as early as days 17-19 days following AI.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES

1. SEQ ID NO: 1-IFNT clone BTP509
MAFVLSLLMALVLVSYGQGRSLGCYLSEDHMLGARENLRLLARMNRLSPHPCLQDRK
DFGLPQEMVEGNQLQKDQAISVLHEMLQQCLNLFYTEHSSAAWNTTLLEQLCTGLQQQ
LEDLDACLGPVMGEKDSDMGRMGPILTVKKYFQGIHVYLKEKEYSDCAWEIIRVEMMR
ALSSSTTLQKRLRKMGGDLNSL
2. SEQ ID NO: 2-clone BTP509
gatccccggaaactagaattcacctgaaggttcacccagaccccatctcagccagcccagcagcagccacatcttccccatggccttcgtg
ctctctctactgatggccctggtgctggtcagctacggccagggacgatctctgggttgttacctgtctgaggaccacatgctaggtgccag
ggagaacctcaggctcctggcccgaatgaacagactctctcctcatccctgtctgcaggacagaaaagactttggtcttcctcaggagatgg
tggagggcaaccagctccagaaggatcaggctatctctgtgctccacgagatgctccagcagtgcctcaacctcttctacacagagcactcg
tctgctgcctggaacaccaccctcctggagcagctctgcactgggctccaacagcagctggaggacctggacgcctgcctgggcccagtg
atgggagagaaagactctgacatgggaaggatgggccccattctgactgtgaagaagtacttccagggtatccatgtctacctgaaagaaa
aagaatacagtgactgcgcctgggaaatcatcagagtggagatgatgagagccctctcttcatcaaccaccttgcaaaaaaggttaagaaa
gatgggtggagatctgaactcactttgagatgactctcgctgactaagatgccacatcaccttcgtacactcacctgtgttcatttcagaag
actctgatttctgcttcagccaccgaaatcattgaattactttaactgatactttgtcagcagtaataagcaagtagatataaaagtactca
gctgtaggggcatgagtccttaagtgatgcctgccctgatgttatctgttgttgatttatgtattccttcttgcatctaacatacttaaaat
attaggaaatttgtaaagttacatttcatttgtacatctattaaaatttctaaaacatgtttaccattttgtgttattaaatttgtcctttg
ttctatttattaaatcaaagaaaatc
3. SEQ ID NO: 3-IFNT
MAFVLSLLMALVLVSYSPGRSLGCYLSENHMLGARENLRLLAQMNRLSTHSCLQDRKD
FGLPWEMVEGDQLQKDQAISVLHEMLQQCFNLFHTEHSSAAWNTTLLEQLCTGLHQQL
DDLDACLGQVMEEKDSALGRMGPILTVKKYFQGIHVYLKKKEYSDCAWEIIRVEMMR
ALSSSTSLQERLRKIGGDLNSS
4. SEQ ID NO: 4-IFNT
atggcttttgtgctctctctactgatggccttggtgctggtcagctacagcccgggacgatctctgggttgttacctgtctgagaaccacat
gctaggtgccagggagaacctcaggctcctggcccaaatgaacagactctccactcattcctgtctgcaagacagaaaagactttggtcttc
cctgggagatggtggagggtgaccagctccagaaggaccaggctatctctgtgctccacgagatgctccagcagtgcttcaacctcttccac
acagagcactcgtctgctgcctggaacaccaccctcctggagcagctctgcactggactccatcagcagctggatgacctggatgcctgcct
ggggcaggtgatggaagagaaagactctgccctgggaaggatgggccccattctgaccgtgaagaagtacttccagggcatccatgtgtac
ctgaaaaagaaggaatacagcgactgcgcctgggaaatcatcagagtggagatgatgagagccctctcttcatcaaccagcttgcaagaa
aggttaagaaagataggtggagatctgaactcatcttga
5. SEQ ID NO: 5-IFNT2
MAFVLSLLMALVLVSYGPGRSLGCYLSEDHMLGARENLRLLARMNRLSPHPCLQDRKD
FGLPQEMVEGNQLQKDQAISVLHEMLQQCLNLFYTEHSSAAWNTTLLEQLCTGLQQQL
EDLDACLGPVMGEKDSDMGRMGPILTVKKYFQGIHVYLKEKEYSDCAWEIIRVEMMR
ALSSSTTLQKRLRKMGGDLNSL
6. SEQ ID NO: 6-IFNT2
tttatttagtttctcatttaattgatatacatttacattgacaaacccaaattttattgggaaaattaaatttctactgtaaaaattaagag
tttagattgactacatttcctaggtcaaacagaaaatatctaactgaaaacacaaacaggaagtgagagagaaattttcggataatgagtac
cgtcttccctatttaaaagccttgcttagaacgatcatcatcagagaacctacctgaaggttcacccagaccccatctcagccagcccagca
gcagccacatcttccccatggccttcgtgctctctctactgatggccctggtgctggtcagctacggcccgggacgatctctgggttgttac
ctgtctgaggaccacatgctaggtgccagggagaacctcaggctcctggcccgaatgaacagactctctcctcatccctgtctgcaggacag
aaaagactttggtcttcctcaggagatggtggagggcaaccagctccagaaggatcaggctatctctgtgctccatgagatgctccagcagt
gcctcaacctcttctacacagagcactcgtctgctgcctggaacaccaccctcctggagcagctctgcactgggctccaacagcagctggag
gacctggacgcctgcctgggcccagtgatgggagagaaagactctgacatgggaaggatgggccccattctgactgtgaagaagtacttcca
gggtatccatgtctacctgaaagaaaaagaatacagtgactgcgcctgggaaatcatcagagtggagatgatgagagccctctcttcatcaa
ccaccttgcaaaaaaggttaagaaagatgggtggagatctgaactcactttgagatgactctcgctgactaagatgccacatcaccttcgta
cactcacctgtgttcatttcagaagactctgatttctgcttcagccaccgaaatcattgaattactttaactgatactttgtcagcagtaat
aagcaagtagatataaaagtactcagctgtaggggcatgagtccttaagtgatgcctgccctgatgttatctgttgttgatttatgtattcc
ttcttgcatctaacatacttaaaatattaggaaatttgtaaagttacatttcatttgtacatctattaaaatttctaaaacatgtttaccat
tttgtgttattaaatttgtcctttgttctatttattaaatcaaagaaaatgagtttctttactcaaaaactttattattattattattatta
aaactttattaaagaatgggtggtt
7. SEQ ID NO: 7-IFNT3
MAFVLSLLMALVLVSYGPGRSLGCYLSEDHMLGARENLRLLARMNRLSPHPCLQDRKD
FGLPQEMVEGSQLQKDQAISVLHEMLQQCFNLFHIEHSSAAWNTTLLEQLCTGLQQQLE
DLDACLGPVMGEKDSDMGRMGPILTVKKYFQDIHVYLKEKEYSDCAWEIIRVEMMRA
LSSSTTLQKRLRKMGGDLNSL
8. SEQ ID NO: 8-IFNT3
ctgaaggttcacccagaccccatctcagccagcccagcagcagccacatcttccccatggccttcgtgctctctctactgatggccctggtg
ctggtcagctacggcccgggacgatctctgggttgttacctgtctgaggaccacatgctaggtgccagggagaacctcaggctcctggccc
gaatgaacagactctctcctcatccctgtctgcaggacagaaaagactttggtcttcctcaggagatggtggagggcagccagctccagaa
ggatcaggctatctctgtgctccacgagatgctccagcagtgcttcaacctcttccacatagagcactcgtctgctgcctggaacaccaccc
tcctggagcagctctgcactgggctccaacagcagctggaggacctggacgcctgcctgggcccagtgatgggagagaaggactctgac
atgggaaggatgggccccattctgactgtgaagaagtacttccaggacatccatgtctacctgaaagaaaaggaatacagtgactgcgcct
gggaaatcatcagagtggagatgatgagagccctctcttcatcaaccaccttgcaaaaaaggttaagaaagatgggtggagatctgaactc
actttgagatgactctcgctgactaagatgccacatcaccttcgtacactcacctgtgttcatttcagaagactctgatttctgcttcagcc
accgaattcattgaattactttagccgatactttgtcagcagtaataagcaagtagatataaaagtactcagctgtaggggcatgagtcctt
aagtgatgcctgccctgatgttatctgttgttgatttatgtattccttcttgcatctaacatacttaaaatattaggatatttgtaaagtta
catttcatttgtacatctattaaaatttctaaaacatgtttaccattttgtgttattaaatttgtcctttgttctatttattaaatcaaaga
aaatgagtttctttactcaaaaactttattattattattaaaactttattaaagaaaaaaaaaaaaaa
9. SEQ ID NO: 9
ENLR
10. SEQ ID NO: 10
LLDR
11. SEQ ID NO: 11
MNRPSPHSCLQDR
12. SEQ ID NO: 12
MDPIVTVK
13. SEQ ID NO: 13
YFQGIHDYLQEK
14. SEQ ID NO: 14
VEMMR
15. SEQ ID NO: 15
ALTSSTTLK
16. SEQ ID NO: 16
LLAR
17. SEQ ID NO: 17
LSPHPCLQDR
18. SEQ ID NO: 18
MGPILTVK
19. SEQ ID NO: 19
YFQGIHVYLK
20. SEQ ID NO: 20
ALSSSTTLQK

### Tables

TABLE 1
Accuracy of Open Cow Test (OCT) when identifying
pregnancy status on Dairy 1 (Tech 3) in Colorado

| D 17 | | % |
|---|---|---|
| Sensitivity | 92/117 | 79 |
| Specificity | 168/206 | 82 |
| False Positive | 38/206 | 18 |
| False Negative | 25/117 | 21 |
| Embryo Mortality | 38/206 | 18 |

| | Pregnant | Total | % |
|---|---|---|---|
| Pregnancy rate OCT | 130 | 323 | 39 |
| Pregnancy rate US | 117 | 323 | 35 |

TABLE 2
Accuracy of OCT when identifying pregnancy status
on day 17 on Dairy 2 (Tech 2) using OCT.

| D 17 | Pregnant OCT + US | Pregnant US | % |
|---|---|---|---|
| Sensitivity | 109 | 151 | 72 |
| | Open OCT + US | Open US | % |
| Specificity | 136 | 154 | 88 |
| | OCT False Pregnant D 17 | Open US | % |
| False Pos | 18 | 154 | 12 |
| | OCT False Open D 17 | Pregnant US | % |
| False Neg | 42 | 151 | 28 |

TABLE 3
OCT Samples

| | OCT | US |
|---|---|---|
| Open | 178 | 154 |
| Pregnant | 127 | 151 |
| Removed | 4 | 4 |
| Total | 309 | 309 |
| Pregnancy rate | 41% | 49% |

TABLE 4
Conserved IFNT Peptide Amino Acid Sequences.

| Sheep | | Bovine | |
|---|---|---|---|
| AA sequence | Mass (Da) | AA sequence | Mass (Da) |
| ENLR (SEQ ID NO: 9) | 530.581 | ENLR (SEQ ID NO: 9) | 530.581 |
| LLDR (SEQ ID NO: 10) | 515.610 | LLAR (SEQ ID NO: 16) | 471.600 |
| MNRPSPHSCLQDR | 1540.735 | MNR | 419.449 |
| (SEQ ID NO: 11) | | | (1584.781) |
| | | LSPHPCLQDR (SEQ | 1165.332 |
| | | ID NO: 17) | |
| K | 146.189 | K | 146.189 |
| MDPIVTVK (SEQ ID | 902.117 | MGPILTVK (SEQ ID | 858.107 |
| NO: 12) | | NO: 18) | |
| YFQGIHDYLQEK (SEQ | 1540.695 | YFQGIHVYLK (SEQ | 1267.493 |
| ID NO: 13) | | ID NO: 19) | |
| VEMMR (SEQ ID | 664.836 | VEMMR (SEQ ID | 664.836 |
| NO: 14) | | NO: 14) | |
| ALTSSTTLK (SEQ ID | 921.059 | ALSSSTTLQK (SEQ | 1035.162 |
| NO: 15) | | ID NO: 20) | |

TABLE 5
Glycosylated Amino Acids in Conserved IFNT
Sequences.
Bovine

| AA sequence | Mass (Da) |
|---|---|
| ENLR (SEQ ID NO: 9) | 530.581 |
| LLAR (SEQ ID NO: 16) | 471.600 |
| MNR | 419.449 |
| LSPHPCLQDR (SEQ ID NO: 17) | 1165.332 |
| K | 146.189 |
| MGPILTVK (SEQ ID NO: 18) | 858.107 |
| YFQGIHVYLK (SEQ ID NO: 19) | 1267.493 |
| VEMMR (SEQ ID NO: 14) | 664.836 |
| ALSSSTTLQK (SEQ ID NO: 20) | 1035.162 |

TABLE 6
Non-glycosylated Conserved Peptide Sequences for Mass
Spectroscopy Identification of IFNT.

| Sheep | | Bovine | |
|---|---|---|---|
| AA sequence | Mass (Da) | AA sequence | Mass (Da) |
| ENLR (SEQ ID NO: 9) | 530.581 | ENLR (SEQ ID NO: 9) | 530.581 |
| K | 146.189 | K | 146.189 |
| VEMMR (SEQ ID NO: 14) | 664.836 | VEMMR | 664.836 |
| | | (SEQ ID NO: 14) | |

TABLE 7
Accuracy estimates comparing pregnancy determination based on
external cervical os antigen concentrations using the bovine
swab device on day 17 to ultrasound determination of pregnancy
on day 32+ following AI (n = 99 lactating dairy cows).

| | Pregnant (OCT + US) | Pregnant (US) | |
|---|---|---|---|
| | (n) | (n) | % |
| Sensitivity | 35 | 37 | 95 |
| | Open (OCT + US) | Open (US) | |
| | (n) | (n) | % |
| Specificity | 18 | 62 | 29 |
| | False Pregnant (OCT) | Open (US) | |
| | (n) | (n) | % |
| False Positive | 44 | 62 | 71 |
| | False Open (OCT) | Pregnant (US) | |
| | (n) | (n) | % |
| False Negative | 2 | 37 | 5 |
| | % | | |
| Negative Predictive | 97 | | |
| Value | | | |

TABLE 8
Accuracy estimates comparing pregnancy determination based
on intra-cervical antigen concentrations using the bovine
swab device on day 17 to ultrasound determination of pregnancy
on day 32+ following AI (n = 99 lactating dairy cows).

| | Pregnant (OCT + US) | Pregnant (US) | |
|---|---|---|---|
| | (n) | (n) | % |
| Sensitivity | 35 | 37 | 95 |
| | Open (OCT + US) | Open (US) | |
| | (n) | (n) | % |
| Specificity | 22 | 62 | 35 |
| | False Pregnant (OCT) | Open (US) | |
| | (n) | (n) | % |
| False Positive | 40 | 62 | 65 |
| | False Open (OCT) | Pregnant (US) | |
| | (n) | (n) | % |
| False Negative | 2 | 37 | 5 |
| | % | | |
| Negative Predictive | 97 | | |
| Value | | | |

TABLE 9
Calculated parameters for open cow test results (external os
or mid cervix) based on pregnancy diagnosis by ultrasound in
66 lactating dairy cows at about 100 DIM after first-survive
timed insemination following the Double OvSynch protocol.

| Endpoints | External Os | Mid Cervix |
|---|---|---|
| TP % (n) | 31.9 (21) | 31.9 (21) |
| TN % (n) | 15.1 (10) | 21.2 (14) |
| FP % (n) | 50 (33) | 43.9 (29) |
| FN % (n) | 3 (2) | 3 (2) |
| ACC (%) (TP + TN/n) | 47 | 53 |
| Se (%) (TP/TP + FN) | 91 | 91 |
| Sp (%) (TN/TN + FP) | 23 | 33 |
| PPV (%) (TP/TP + FP) | 39 | 42 |
| NPV (%) (TN/TN + FN) | 83 | 88 |
| EM (%) (FP/TP + FP + FN) | 59 | 56 |

Sp = Specificity,
Se = Sensitivity,
TP = True Positive,
TN = True Negative,
FP = False Positive,
FN = False Negative,
ACC = Accuracy,
EM = Estimated Embryonic Mortality

TABLE 10
Calculated parameters for open cow test results based on pregnancy diagnosis
by ultrasound in beef heifers and cows after insemination or embryo transfer.

| | Beef Heifers | Beef Heifers | Beef Cows | Beef Cows |
|---|---|---|---|---|
| Endpoints | No AI (n = 19) | AI (n = 102) | AI (n = 135) | ET (n = 126) |
| TP % (n) | 0 (0) | 22.5 (23) | 55.6 (75) | 26.2 (33) |
| TN % (n) | 100 (19) | 64.7 (66) | 34.8 (47) | 63.5 (80) |
| FP % (n) | 0 (0) | 3 (3) | 3.7 (5) | 8.7 (11) |
| FN % (n) | 0 (0) | 9.8 (10) | 5.9 (8) | 1.6 (2) |
| ACC (%) (TP + TN/n) | 100 | 87 | 90 | 90 |
| Se (%) (TP/TP + FN) | — | 70 | 90 | 94 |
| Sp (%) (TN/TN + FP) | 100 | 96 | 90 | 88 |
| PPV (%) (TP/TP + FP) | — | 88 | 94 | 75 |
| NPV (%) (TN/TN + FN) | 100 | 87 | 85 | 98 |
| EM (%) (FP/TP + FP + FN) | — | 8 | 6 | 24 |

Sp = Specificity,
Se = Sensitivity,
TP = True Positive,
TN = True Negative,
FP = False Positive,
FN = False Negative,
ACC = Accuracy,
EM = Estimated Embryonic Mortality
Sp = Specificity,
Se = Sensitivity,
TP = True Positive,
TN = True Negative,
FP = False Positive,
FN = False Negative,
ACC = Accuracy,
EM = Estimated Embryonic Mortality

1. A method of determining pregnancy and identifying open cows, comprising:
breeding a cow;
obtaining a genital tract sample from the cow;
detecting a level of an interferon tau (IFNT) polypeptide in the genital tract sample;
identifying the cow as pregnant if the level of IFNT is higher than a reference control or as not pregnant if the level of IFNT is the same as or lower than the reference control; and
performing additional breeding of the cow if the cow is determined to be not pregnant.

2. The method of claim 1, wherein the genital tract sample is obtained from the cow on or before day 18 post breeding.

3. The method of claim 1, wherein the genital tract specimen is obtained from the open cow on day 15, 16, 17, or 18 post breeding.

4. The method of claim 1, wherein the method identifies the open cow on or before day 18 post breeding.

5. The method of claim 1, wherein the methods identifies the open cow on day 15, 16, 17, or 18 post breeding.

6. The method of claim 1, wherein the level of the IFNT polypeptide is detected by an immunoassay.

7. The method of claim 6, wherein the immunoassay comprises enzyme-linked immunoassay (ELISA), lateral flow immunosorbent assay, radioimmunoassay, or a derivative thereof.

8. The method of claim 6, wherein the immunoassay is performed by contacting the genital tract specimen with an antibody targeting a glycosylated IFNT polypeptide.

9. The method of claim 8, wherein the glycosylated IFNT polypeptide comprises a bovine IFNT polypeptide.

10. The method of claim 8, wherein the glycosylated IFNT polypeptide comprises a glycosyl molecule bound to an amino acid of the IFNT polypeptide.

11. The method of claim 10, wherein the amino acid is located at position 101 of the IFNT polypeptide.

12. The method of claim 1, wherein the method detects an IFNT polypeptide comprising SEQ ID NO: 1, SEQ ID NO: 3, SEQ ID NO: 5, SEQ ID NO: 7, or a variant thereof.

13. The method of claim 1, further comprising detecting a level of an additional RNA or polypeptide.

14. The method of claim 1, wherein the additional RNA or polypeptide is interferon-stimulated gene 15 (ISG15) polypeptide in the genital tract sample.

15. The method of claim 1, further comprising detecting a level of progesterone in a genital tract sample, blood sample, or a milk sample.

16. A method of discriminating between a pregnant cow and an open cow, the method comprising:
breeding a cow;
obtaining a genital tract sample from the cow;
measuring a level of an interferon tau (IFNT) polypeptide in the genital tract sample;
detecting a high IFNT level in the pregnant cow relative to a reference control; and
detecting a low IFNT level in the open cow relative to a reference control, wherein the open cow is subjected to an additional round of breeding.

17.-30. (canceled)

31. The method of claim 1, wherein the genital tract sample is collected using a swab device.

32. The method of claim 31, wherein the swab device is a double-guarded uterine swab device.

33. The method of claim 31, wherein the swab device is enclosed in a retractable sheath.

34. The method of claim 31, wherein the swab device is twirled for about 30 seconds to collect the genital tract sample.

35. The method of claim 31, wherein the swab device is saturated with cervical/vaginal fluid and then it is retracted into a plastic sheath before the swab is removed from the genital tract.

36. The method of claim 1, wherein the genital tract sample is collected from the external os of the cervix.

37. The method of claim 1, wherein the genital tract sample is collected from an internal cervical swab.
